(19) United States
(12) Patent Application Publication
(10) Pub. No.: US 2018/0110466 A1
(43) Pub. Date: Apr. 26, 2018

(54) Apparatus and Method for Multivariate Impact Injury Risk and Recovery Monitoring

(71) Applicant: IMPAXX Solutions, Inc., Portola Valley, CA (US)

(72) Inventor: John D. Ralston, Portola Valley, CA (US)

(73) Assignee: IMPAXX Solutions, Inc., Portola Valley, CA (US)

(21) Appl. No.: 15/795,027

(22) Filed: Oct. 26, 2017

Related U.S. Application Data
(60) Provisional application No. 62/560,969, filed on Sep. 20, 2017.
(60) Provisional application No. 62/553,886, filed on Sep. 3, 2017.
(60) Provisional application No. 62/544,905, filed on Aug. 14, 2017.
(60) Provisional application No. 62/437,951, filed on Dec. 22, 2016.
(60) Provisional application No. 62/412,979, filed on Oct. 26, 2016.

(51) Int. Cl. A61B 5/00; A61B 5/0478

(52) U.S. Cl. CPC A61B 5/6814; A61B 5/6832; A61B 5/4064; A61B 5/4076; A61B 5/0478; A61B 5/7275

(57) ABSTRACT

An apparatus has a housing adapted for mechanical coupling with a skull of a human. A first sensor is positioned in the housing to collect linear motion signals. A second sensor is positioned in the housing to collect rotational motion signals. A processor is positioned in the housing connected to the first sensor and the second sensor. The processor is configured to process the linear motion signals and the rotational motion signals to derive a cumulative impact power measure of repetitive sub-concussive head impacts. The cumulative impact power measure is compared to a threshold indicative of the onset of neural tissue deformations corresponding to physiological changes from repetitive sub-concussive head impacts. An alert is supplied when the cumulative impact power measure is proximate the threshold.

### CROSS-REFERENCE TO RELATED APPLICATION

This application claims priority to U.S. Provisional Patent Application Ser. No. 62/412,979, filed Oct. 26, 2016, U.S. Provisional Patent Application Ser. No. 62/437,951, filed Dec. 22, 2016, U.S. Provisional Patent Application Ser. No. 62/544,905, filed Aug. 14, 2017, U.S. Provisional Patent Application Ser. No. 62/553,886, filed Sep. 3, 2017, and U.S. Provisional Patent Application Ser. No. 62/560,969, filed Sep. 20, 2017, the contents of each such application is incorporated herein by reference.

### FIELD OF THE INVENTION

This invention relates generally to electronic sensors. More particularly, this invention is directed toward sensors for multivariate impact injury risk and recovery monitoring.

### BACKGROUND OF THE INVENTION

Participation in athletic activities, military training and deployments, and a variety of industrial workplace activities often exposes the participants to risks of physical injuries that can be caused by both direct and indirect mechanical impacts to the head and other parts of the body. These impacts can cause rapid accelerations and decelerations of many different body parts, including: the head, neck, and brain; torso and internal organs; limbs and extremities such as arms, legs, hands, and feet, including the corresponding muscles and nerves; and joints such as the knees, ankles, elbows, wrists, shoulders, including the corresponding bones, ligaments, tendons, and cartilage. Linear and rotational motion transfers mechanical energy from the outside environment to the various body parts for the duration of the impact. For example, a knee joint forced inward may result in a medial collateral ligament sprain, a knee joint forced outward may result in a lateral collateral ligament injury, a violent knee rotation with the foot in a fixed position may sprain ligaments in the center of the knee joint and hyper-extension of a leg may lead to a cruciate collateral and capsular ligament sprain. The impact forces giving rise to linear motion and impact torques giving rise to rotational motion, coupled with the resulting linear and rotational displacements, determine the amount of energy transferred to a specific body part. This energy, together with the duration of the impact, determines the power transferred to the body part.

Above certain thresholds, which may vary from user to user and for different body parts, the transferred power can lead to physiological changes that cause both reversible and permanent injuries spanning a broad continuum in terms of their extent and severity. The extent of the damage may be localized to the site of the mechanical impact, also referred to as a focal injury, or it may affect a much wider volume, also referred to as a diffuse injury. The severity of the injury may vary from localized cellular damage to torn tissues, ruptured blood vessels, bleeding, and broken bones. Head impacts may also lead to a wide range of additional short-term and long-term “concussion” symptoms that can be observed in a clinical examination as changes in cognitive functions (degraded attention and memory), motor functions (impaired coordination and balance), sensory functions (damage to vision, speech, hearing), and emotional and behavioral conditions (depression, anxiety, aggression, impulse control, mood, and other personality traits).

Until recently, it was widely proposed that most concussions are temporary and reversible injuries, and that most individuals who sustain a concussion eventually achieve a full recovery, since the observable symptoms eventually returned to their pre-injury status or baseline levels. However, it is now understood that underlying physiological changes may be present even in the absence of any diagnosed symptoms in the first place. It is also now recognized that head impacts can initiate complex molecular and metabolic pathologies, along with neurotoxic and neuro-inflammatory reactions, which may lead to longer term neurodegenerative disorders.

The various injuries described above may be the result of a single impact event, or may be caused by cumulative physiological changes from multiple impacts over time, and the corresponding physiological change thresholds may differ. The various physiological changes and observed symptoms arising from impact injuries may not all be present at the time of injury, but may instead evolve over time. Certain injuries may be risk factors for later conditions, triggering or contributing to progressive deterioration or long term degenerative consequences. For example, repetitive impacts to the knees, hips, and ankles are a major risk factor for the development of post-traumatic osteoarthritis many years post-injury, often leading to severely limited mobility due to joint pain, swelling, and fluid accumulation that result from deterioration of cartilage and bones. Repetitive head impacts are now known to be a significant risk factor for chronic traumatic encephalopathy (CTE), a debilitating degenerative disease of the brain.

Because of the wide range of cognitive, motor, and sensory impairments that can arise from repetitive head impacts, as well as changes in mood, behavior, and personality, this issue is now recognized as an enormous public health challenge. Although much attention has been directed specifically at the issue of concussion injuries, a significant volume of evidence has revealed that physiological changes in the brain resulting from the accumulation of many small direct or indirect head impacts, none of which on their own trigger any concussion symptoms, also lead to neurological injuries and long-term degenerative neural disorders, and significantly increase the head impact injury risk pool. Because of this evidence, concussions are beginning to be viewed not as a single distinct class of injury, but as one segment of a wide and continuous spectrum of cumulative head impact injuries that all trigger some level of axonal damage, often referred to as diffuse axonal injury (DAI). This spectrum of injuries is characterized by highly heterogeneous and multifactorial disorders, complex and diverse pathological changes that may continue for months or years, and some epidemiological support showing associations with other classical chronic neurodegenerative disorders such as Parkinson's Disease, Alzheimer's Disease, Multiple Sclerosis (MS), amyotrophic lateral sclerosis (ALS), and Chronic Traumatic Encephalopathy (CTE). The prevalence of CTE in subjects with no history of concussion suggests that sub-concussive hits are sufficient to lead to the development of CTE, and it has recently been argued that it is the chronic and repetitive nature of head trauma, irrespective of concussive symptoms, that is the most important driver of disease.

A variety of wearable devices have been developed and utilized in attempts to predict the occurrence of concussion injuries based on measured impact biomechanics such as the number of impacts or the linear and rotational head acceleration or velocity. None of these tools, however, are effective at monitoring and limiting brain injury risks arising from exposure to head impacts accumulated over time, in the absence of any reported or diagnosed concussion symptoms.

Given the above complexities and potential long-term risks of impact injuries, there is a need for more effective means of assessing physiological changes and injury severity to the head and other body parts at the time of impact, and to track cumulative injury risks over time to improve injury prevention, diagnosis, and treatment, as well as provide objective support for remove-from-play and return-to-play decisions. The present invention enables significant advances and improvements in real-time monitoring of impact injury risks and recovery by providing guidance to users and supervisory personnel when a user should be removed from activity to avoid onset or further accumulation of transient or permanent physiological changes that are indicative of potential injury or progressive deterioration due to exposure to mechanical impacts. The present invention also provides guidance to users and supervisory personnel when a user can return to activity following sufficient recovery from transient physiological changes.

### SUMMARY OF THE INVENTION

An apparatus has a housing adapted for mechanical coupling with a skull of a human. A first sensor is positioned in the housing to collect linear motion signals. A second sensor is positioned in the housing to collect rotational motion signals. A processor is positioned in the housing connected to the first sensor and the second sensor. The processor is configured to process the linear motion signals and the rotational motion signals to derive a cumulative impact power measure of repetitive sub-concussive head impacts. The cumulative impact power measure is compared to a threshold indicative of the onset of neural tissue deformations corresponding to physiological changes from repetitive sub-concussive head impacts. An alert is supplied when the cumulative impact power measure is proximate the threshold.

### BRIEF DESCRIPTION OF THE DRAWINGS

Preferred and alternative examples of the present invention are described in detail below with reference to the following drawings.

FIG. 1 shows histograms of (a) linear, (b) rotational, and (c) total (linear+rotational) impact power transferred to the brain for nine female soccer athletes throughout a 3-month season, along with (d) a summary of calculated head impact powers.

FIG. 2 shows the histogram of daily total cumulative impact powers for all athletes throughout 3-month soccer season.

FIG. 3 illustrates the high-angular-resolution diffusion spectrum MM (DSI) imaging techniques developed to detect microscopic damage to brain tissues.

FIG. 4 presents DSI images showing local differences in white matter primary diffusion direction between players and age matched controls for two different statistical significance levels: (a) p<0.005); (b) p<0.0005.

FIG. 5 shows the calculated number of outlier voxels in each player's mid-season DSI scan, plotted vs. the maximum cumulative daily impact dose, and the total cumulative impact power measured over 1-day and 2, 3, and 4-week periods immediately preceding each player's mid-season DSI scan.

FIG. 6 shows changes in MDA0 WM diffusion observed in all 3 in-season scans are significantly larger than age/gender-matched controls in all four WM compartments into which the brain images have been partitioned.

FIG. 7 shows median % MDA0 change between scans 3 and 4 plotted as a function of the maximum daily total impact power received by each player in the in-season period between scans 2 and 3, for each of the four WM compartments into which the brain images have been partitioned.

FIG. 8 shows in-vivo MDA/DSI observations of localized damage at cortical sulci, consistent with the predicted localized stress enhancement at these structures.

FIG. 9 illustrates impact monitoring device (IMD) calibration using the number of DSI outlier voxels observed as a function of total cumulative impact power transferred to the brain.

FIG. 10 illustrates the preferred form factor and methods of attachment for the IMD according to the invention.

FIG. 11 is a block diagram of an IMD according to the invention.

FIG. 12 illustrates a preferred embodiment of an IMD electronics module and enclosure according to the invention.

FIG. 13 illustrates IMD miniaturization using functional partitioning on a folded flexible printed circuit substrate.

FIG. 14 illustrates IMD thickness minimization using embedded die within a flexible circuit substrate.

FIG. 15 illustrates IMD thickness minimization using laminated cover layers and planarizing cover layer adhesive layers.

FIG. 16 illustrates a two-section IMD in which one section houses the electronic components and the other section houses (a) a cylindrical Li-ion battery, or (b) a rectangular Li-polymer battery.

FIG. 17 shows photographs of a two-section IMD worn on (a) the user's head, and (b) the user's knee.

FIG. 18 illustrates an IMD with integrated adhesive.

FIG. 19 is a diagram illustrating IMD device deployment and data flow for on-sensor calculation of impact power transferred to the brain, comparison with injury risk thresholds, and setting/transmission of alarms, all in real time.

FIG. 20 illustrates the derivation of the moments of inertia of the brain used by the IMD to calculate power transferred to the brain during impacts.

FIG. 21 is a graphical representation of linear and rotational head impact power (a) for a single impact; (b) accumulated over multiple impacts; (c) combined status for both single impacts and cumulative impacts over an extended time period.

FIG. 22 illustrates the graphical dosimeter presentation on a mobile device of cumulative head impact power status for a team of athletes or other users.

FIG. 23 illustrates IMD sensors being utilized with additional system components to control, configure, or manage multiple sensors, manage user and team rosters, assign sensors to specific users, download and display impact data, and transfer data from sensor devices to computer or cloud-based data storage, analytics, and reporting components.

FIG. 24 illustrates a dosimeter charger/long-range wireless receiver configured as a clip-on module designed to be worn on the belt or clipped to a shirt pocket of supervisory personnel.

FIG. 25 illustrates postural sway measurements on a force plate.

FIG. 26 illustrates measurement of medial-lateral (ML) and anterior-posterior (AP) body sway on a force plate with user in a variety of stances.

FIG. 27 presents sample postural sway data from force plate and body-worn accelerometer, and a list of metrics derived from such data to quantify various disorders.

FIG. 28 illustrates variations in the step-to-step and stride-to-stride regularity of medial-lateral (ML), anterior-posterior (AP), and vertical (V) head accelerations, which can serve as a sensitive complementary measure of motion disorders when the user is in motion.

FIG. 29 shows (a) process flow for analysis of real-time gait cycle parameters; and (b) comparison of gate cycle parameters for healthy individuals and PD patients.

FIG. 30 illustrates sample postural sway data from an IMD, along with calculated total sway power vs. time for sub-concussive head impact exposure, recorded for six high school football players over a two-week period.

FIG. 31 presents data demonstrating a larger total sway path length observed for head-mounted vs. waist-mounted accelerometers.

FIG. 32 illustrates visual processing regions in the brain and systems to monitor eye-tracking.

FIG. 33 shows sample eye-tracking data before and after a concussion.

FIG. 34 illustrates eye-tracking data corrected for head motion due to impaired balance.

FIG. 35 illustrates head-mounted systems integrating sensors for cumulative head impact monitoring, postural sway measurements, and corrected eye-tracking measurements.

FIG. 36 illustrates a mobile system for measurements of cumulative head impact loads, postural sway, corrected eye-tracking, and visual attention deficits.

FIG. 37 illustrates the locus of human visual attention within images.

FIG. 38 illustrates individual visual fixation episodes linked by a series of saccades.

FIG. 39 illustrates human visual fixation on images of human faces.

FIG. 40 illustrates human visual attention in more complex images.

FIG. 41 illustrates process steps for the utilization of eye-tracking data to produce a threshold binary map that encompasses a fractional area of an image containing the most visually relevant regions.

FIG. 42 shows measured histogram of the radii of regions of interest (ROI) centered about each fixation location in an image.

FIG. 43 illustrates images with one central object and images with multiple objects and textures.

FIG. 44 illustrates images containing universally recognized objects such as faces, people, body parts, animals, text, and cars, and combining multiple low-level visual features.

FIG. 45 illustrates an eye-tracking calibration procedure in which the user's attention is directed to 5 points on the screen.

FIG. 46 illustrates visual attention of a viewer within a static image.

FIG. 47 illustrates pre-exposure and post-exposure binary visual relevance maps.

FIG. 48 illustrates differences between pre-exposure and post-exposure visual relevance maps for users exposed to sub-concussive head impact loads, and for control subjects.

FIG. 49 illustrates visual attention of a viewer within a series of moving images.

FIG. 50 illustrates a system configured in accordance with an embodiment of the invention.

### DETAILED DESCRIPTION

Diffusion Tensor Imaging (DTI) and functional magnetic resonance imaging (fMRI) research have both shown that repeated head impacts, from a single season of soccer or football for example, result in objectively measurable brain damage in the absence of diagnosed concussion symptoms. The current invention has been used to show that routine sub-concussive impacts in many athletic activities can transfer mechanical powers in the range 0.25 kW-5 kW per impact from the outside environment to an athlete's brain. In parallel, high-angular-resolution diffusion spectrum MRI (DSI) imaging and voxel-wise multi-dimensional anisotropy (MDA) techniques have been developed, and their application has revealed that the accumulation of multiple sub-concussive head impacts at these levels during athletic training and competition generates observable transient and persistent physiological changes to the brain, even in the absence of any reported or diagnosed concussion symptoms. These physiological changes indicate the presence of both localized and diffuse damage to neural cells and tissues, as well as damage that can contribute to progressive deterioration over time.

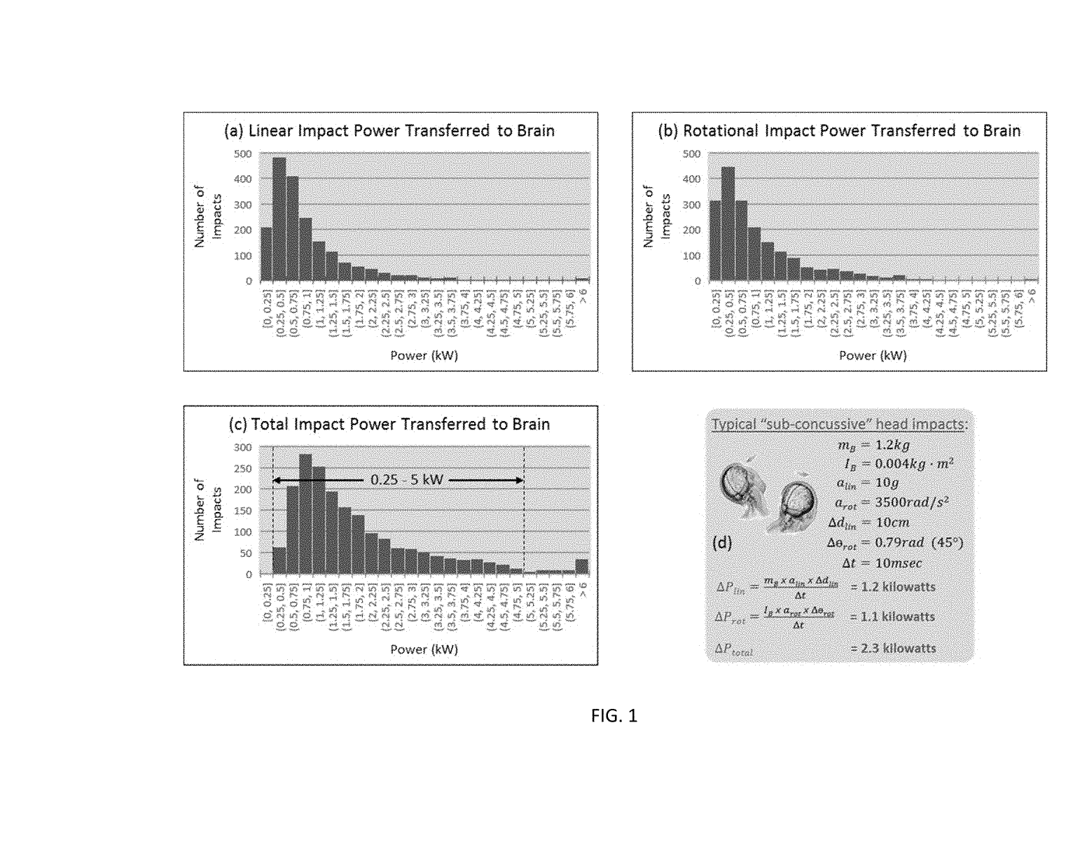

FIG. 1 shows sample histograms of (a) linear, (b) rotational, and (c) total (linear+rotational) impact power transferred to the brain, as measured using Impact Monitoring Devices (IMDs) configured in accordance with embodiments of the invention. The IMDs are sometimes referred to herein as dosimeters. While a dosimeter is a device worn by an individual to measure the individual's exposure to radiation, the term is appropriated here, but in the context of a device worn by an individual to measure the individual's exposure to head trauma. The IMD is also sometimes referred to as a cumulative impact power measuring device.

The data in FIG. 1 is for each of nine female soccer athletes throughout a 3-month season, along with (d) a summary of calculated head impact powers. A total of 1938 head impacts were registered by the nine athletes over the 11 weeks for which data was recorded, corresponding to an average of 215 head impacts per player over the season (low=95, high=327), or ˜20 impacts per player per week. Although the relative magnitude of linear vs. rotational power varied widely across individual impacts, the histograms for linear and rotational impact power components were similar, with both peaked between 0.25 kW and 0.75 kW. The total impact power distribution peaks between 0.5 kW and 1.5 kW per impact, and reveals that routine sub-concussive head impacts due to headers, tackles, collisions, and falls transfer between 0.25 kW and 5 kW from the external environment to an athlete's brain, with few impacts registering beyond 5 kW. The individual impact values reported here are all significantly lower than those reported to have a high probability of causing a concussion, and none of the players for whom data is shown in FIG. 1 were diagnosed with any concussion symptoms.

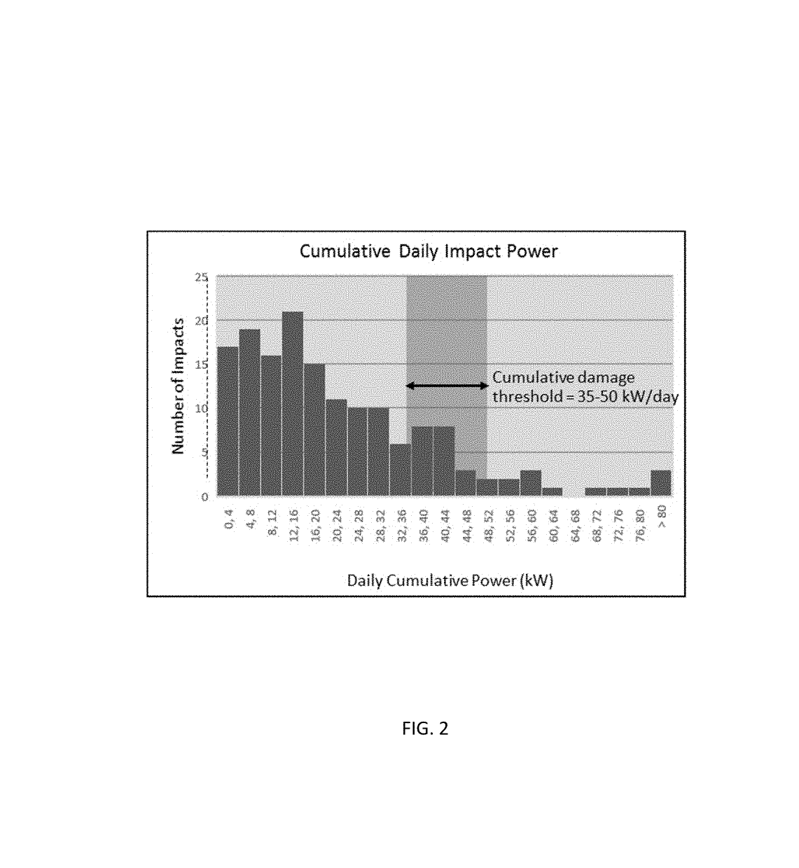

A shown in FIG. 2, the cumulative daily impact power distribution reveals routine daily impact loads extending out to 60 kW, with 6 players receiving daily impact loads between 68 kW and 110 kW.

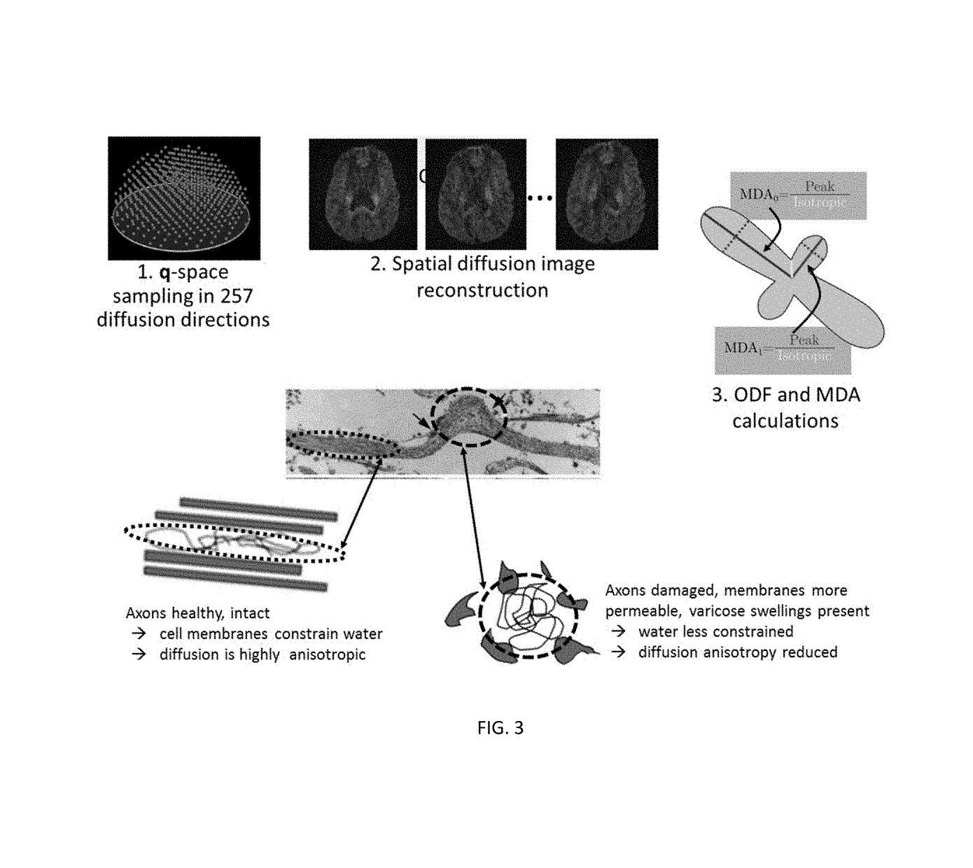

FIG. 3 illustrates the basic principle and key process steps for the high-angular-resolution diffusion spectrum DSI imaging techniques developed to detect microscopic damage to brain tissues. The basic principle is as follows. While the whole brain suffers dynamic tissue deformation during head impacts, it is the axons, the long transmission lines of the white matter (WM), that suffer the most pronounced damage, with their internal microtubule structures mechanically breaking. This leads to the signature varicose swellings along the axons. The DSI scanning techniques developed in this invention enable very sensitive and high-resolution imaging of changes in the diffusion of water within the axons. When the axons are healthy and intact, the diffusion is very anisotropic, because the water is constrained to move within the cell membranes. When the axons become damaged, their membranes more permeable, and varicose swellings appear, the water is no longer as well constrained, and the observed diffusion anisotropy is reduced.

In the present invention, MRI diffusion weighted images (DWI) using diffusion spectrum image (DSI) sampling at 2.0×2.0×2.0 mm³ resolution were acquired using a Siemens 3T Prisma scanner, for athletes exposed to sub-concussive head impacts, at multiple time points throughout the season: scan 1 at the beginning of the season, scan 2 in the middle of the season, scan 3 at the end of the season, and scan 4 three months after the season was over (washout period and pseudo-baseline). As a control group, four DSI scans were also acquired over the same period for age- and gender-matched healthy subjects without high risks for head trauma or history of concussion. High resolution (0.94 mm³) isotropic T1 and T2 weighted scans were also acquired for all subjects. Each diffusion dataset consisted of a series of images in 257 diffusion directions, from which spatial diffusion images were reconstructed using the generalized q-ball imaging (GQI) algorithm as implemented in DSI-Studio, following which generalized fractional anisotropy (GFA) images were extracted. Multimodal spatial normalization (ANTs) was applied to previously skull stripped, aligned, and distortion corrected T1 and T2 weighted volumes, which were previously rigidly registered to the subject's GFA volume. The ANTs symmetric group-wise normalization (SyGN) method was utilized to construct a custom multimodal population-specific brain template of all subjects, centered in MNI space. For each diffusion scan, primary (MDA0), second order (MDA1), and third order (MDA2) anisotropy values were calculated at each WM voxel and then spatially normalized at an isotropic spatial resolution of 1 mm³. MDA values have been shown analytically and experimentally to be an important alternative to widely utilized fractional anisotropy (FA) or GFA measures of WM because they provide information about diffusivity in more than one direction, and can thus provide superior accuracy and differentiation of the underlying white matter structure and account for anisotropy in regions with multi-directional fiber crossings. Both local and global diffusion changes within brain WM were assessed for the players vs. the controls.

To explore local changes of primary anisotropy (MDA0), each player's and control's end-of-season scan (scan 3) was normalized (as a % change) with the same participant's out-of-season washout scan (scan 4). Differences in these normalized scan-3 MDA0 values between players and controls were estimated in SPM12 with an unpaired t-test with exploratory statistical significance thresholds of p<0.005 and p<0.0005).

To estimate global changes of MDA0 and MDA1 diffusion, a 99.9% confidence limit of expected diffusion values was defined from the out-of-season washout scan (scan 4) at each voxel; this procedure was done separately for the players and controls. The numbers of voxels in WM surpassing the expected limit were then summed for each player and control in each of their respective scans 1, 2 and 3. A t-test was used to determine if there was a significantly greater than expected number of outlier values of MDA0 or MDA1 in the players for scans 1, 2 and 3.

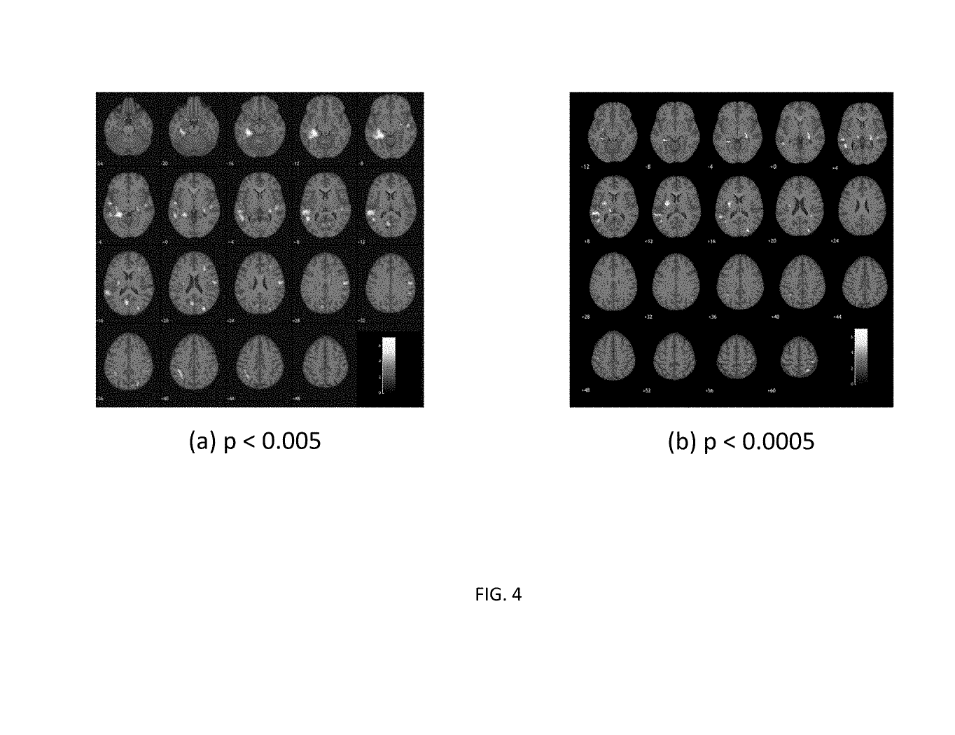

FIG. 4 presents DSI images showing local differences in white matter primary diffusion direction between players and age matched controls for two different statistical significance levels: (a) p<0.005); (b) p<0.0005. Changes in local WM diffusion between end-of-season images (MDA0 scan 3) normalized by the out-of-season washout/pseudo-baseline image (scan 4) were calculated for each subject and compared with similarly normalized scans for the age-matched controls. As shown in FIG. 4, clusters of WM changes are observed for the players vs. controls in both deep WM and at the white matter-cortical border, including at the cortical sulci.

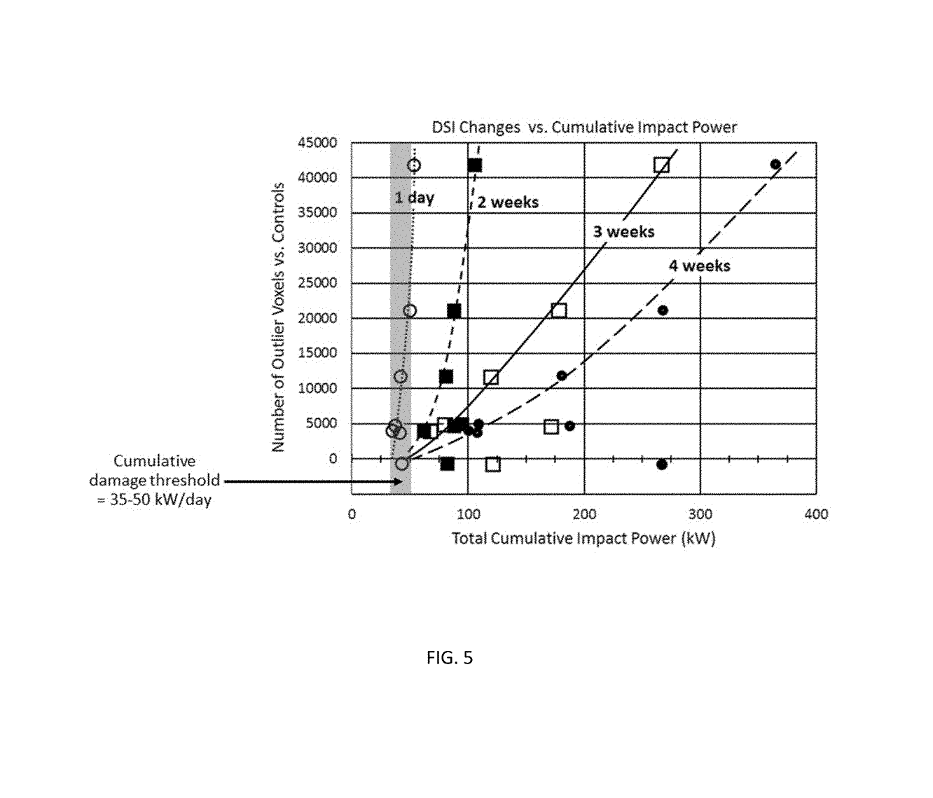

When the number of outlier voxels in the above WM clusters was plotted as a function of the maximum cumulative daily impact dose, along with the total cumulative impact power measured over the 1-day and 2, 3, and 4-week periods immediately preceding each player's mid-season scan, the data exhibited a non-linear relationship, as shown in FIG. 5, with a pronounced threshold behavior for the onset of outlier voxels. The cumulative power threshold above which outlier voxels are observed is on the order of 35-50 kW, which falls within the range of typical cumulative daily impact loads for all athletes in this study. The differences between the results for 1 day and 2, 3, and 4 weeks indicate that a significant fraction of the observed outlier voxel groupings emerge and persist for varying periods of time following impact exposure, and that some fraction then begins to dissipate. Further analyses revealed that accumulated daily exposure doses <50 kW initially triggered transient physiological changes in the brain WM, whereas accumulated daily exposure doses above 100 kW triggered persistent WM changes, or WM changes that required longer recovery times. As can be seen in FIG. 2, these threshold values fall within the range of routine daily head impact loads for the athletes, highlighting the importance of taking preemptive action to avoid more serious injuries accumulating over time.

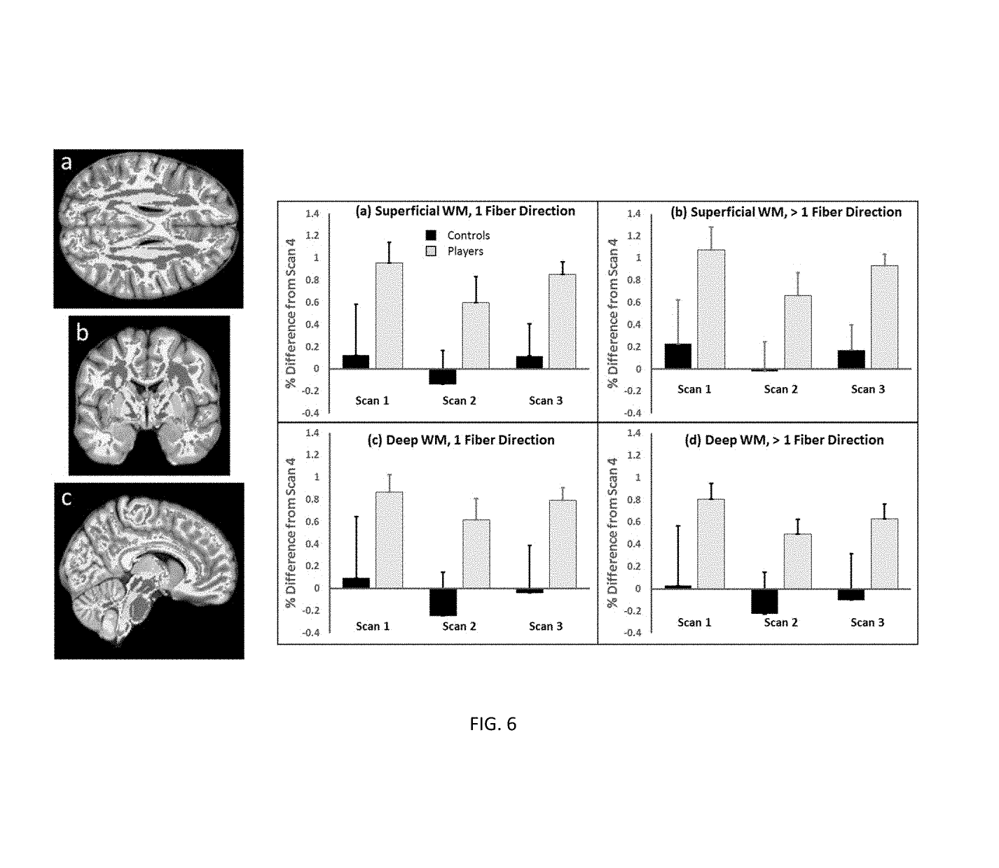

To test the hypotheses that global impact-related diffusion changes might demonstrate a regional susceptibility (deep vs. superficial WM) or local structural susceptibility (single vs. multiple fiber crossings), we partitioned the white matter into four compartments. To do this, we first processed 467 diffusion scans from the Human Connectome Project data set as described above. From this, we created an independent probability map indicating whether a white matter voxel was most likely (>70% of the population) to consist of only a single fiber track (MDA0 but no MDA1 or MDA2 above noise threshold) or to consist of more than a single fiber track (MDA1 and MDA2 above noise threshold). These two compartments were then divided into those WM voxels that were within 2 mm of the gray matter (superficial WM) and those that were deeper than 2 mm from the gray matter (deep WM). These four compartments are shown in FIG. 6. To explore changes of primary anisotropy, mean MDA0, MDA1, MDA2 values in each of these four compartments were extracted from each player's and control's scans. To account for individual differences of diffusion, each of the regional MDA values from in-season scans (scans 1,2, and 3) were normalized (as a % change) with the same participant's out-of-season washout scan (scan 4). Differences in the normalized MDA values between players and controls were modeled by analysis of variance in the software package R with scan (1,2,3), group (player or control), WM region (superficial or deep), or local WM structure (1 or >1 fiber directions).

The MDA0 data was utilized first to test for a group effect (players vs. controls), as well as potential WM region (superficial or deep) or local WM structure (1 or >1 fiber directions) interactions. We ran a fully factored 4-way ANOVA based on subject-specific averaged MDA0 percentage difference from scan 4, averaging within each of the three in-season scans and within each of the two WM regions and two WM fiber crossing categories. After dropping insignificant terms, group (players vs. controls) was the only remaining significant effect (F=41.23 on 1 and 206 df, p<1e-09). FIG. 6 summarizes the diffusion differences between players and controls, and shows that these differences were present across all 3 scans in all four WM compartments. No significant dependence of the MDA0 diffusion changes on WM region or structure were observed across the four compartments. Similar hypotheses were tested for % difference of MDA1 and MDA2 diffusion values, with the caveat that these 2 regions are not differentiated in terms of local WM structure (by definition, MDA1 and MDA2 values above a noise threshold only occur in voxels where >1 fiber tracts cross). Similar results were observed as for MDA0, with a near significant group effect for MDA1 (F=3.36 on 1 and 102 df, p<0.07) and for MDA2 (F=4.654 on 1 and 102 df, p<0.034), but no significant WM region or structure interactions across the four compartments.

These results demonstrate a significant difference of diffusion anisotropy throughout the white matter of soccer players throughout the season (scans 1, 2, and 3), compared with the pseudo-baseline values observed following the 4-month washout period. For the controls, none of the in-season (scan 1, 2, or 3) MDA values were statistically different from the corresponding washout values (scan 4). The diffusion changes observed for the players are similar across all four WM compartments, and show no statistically significant differences in the degree of change between superficial vs. deep WM, or between WM regions with different local structure (1 or >1 fiber directions).

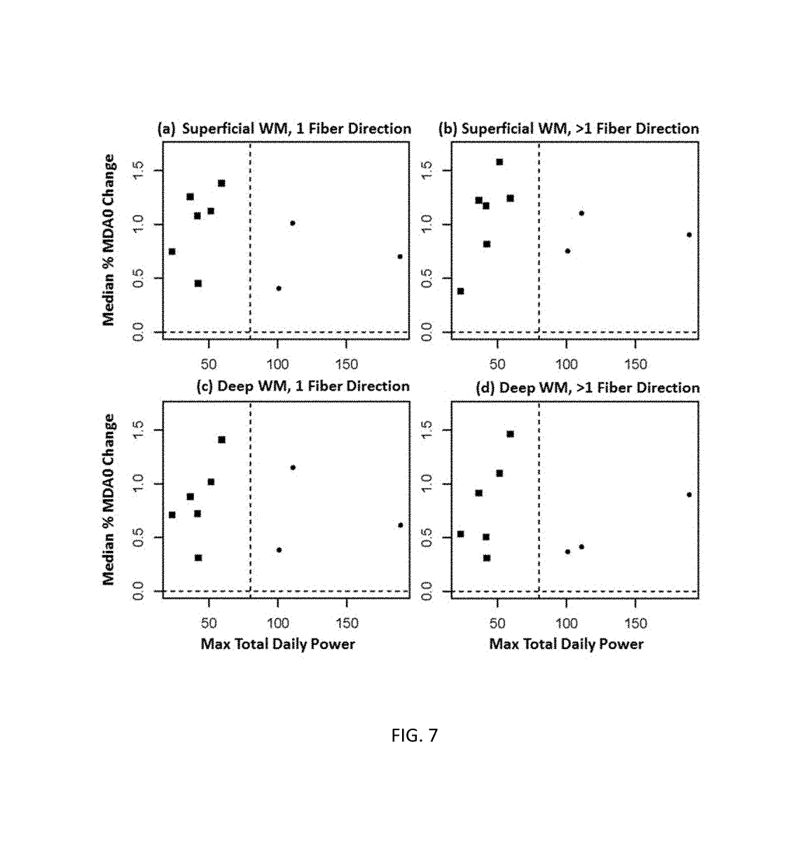

The MDA0 data was next utilized to investigate the hypothesis that lower accumulated exposure doses may initially trigger transient physiological changes in the brain WM, whereas accumulated exposure doses above an observable threshold may trigger persistent WM changes, or WM changes that require a longer recovery time. FIG. 7 shows median % MDA0 change between scans 3 and 4 plotted as a function of the maximum daily total impact power received by each player in the in-season period between scans 2 and 3. For each of the four WM compartments, the % MDA changes are plotted for all nine players. The six athletes represented by the black squares all exhibit diffusion changes that overall show increasing patterns with the athlete-specific maximum daily total cumulative sub-concussive impact power exposure up to 50 kW between scans 2 and 3. The median MDA0 percent change values are positive for all nine athletes, representing a consistent pattern of median decreases in MDA0 values between scans 3 and 4. Since the MDA0 values are related to the magnitude of the in-season diffusion changes, we hypothesize that these consistent decreases for all nine players may be due to a post-season wash-out recovery process, although larger studies are necessary to investigate this hypothesis further. In stark contrast to the linear pattern exhibited by the first six athletes (squares), the three athletes represented by black dots were all exposed to a maximum daily total cumulative sub-concussive impact power exceeding 100 kW between scans 2 and 3, and all three athletes show significantly less washout of the resulting diffusion changes at scan 4. These results indicate that a threshold may exist for maximum daily total cumulative sub-concussive impact power below which physiological changes in the WM are predominantly transient, but above which these changes become persistent for at least 3 months.

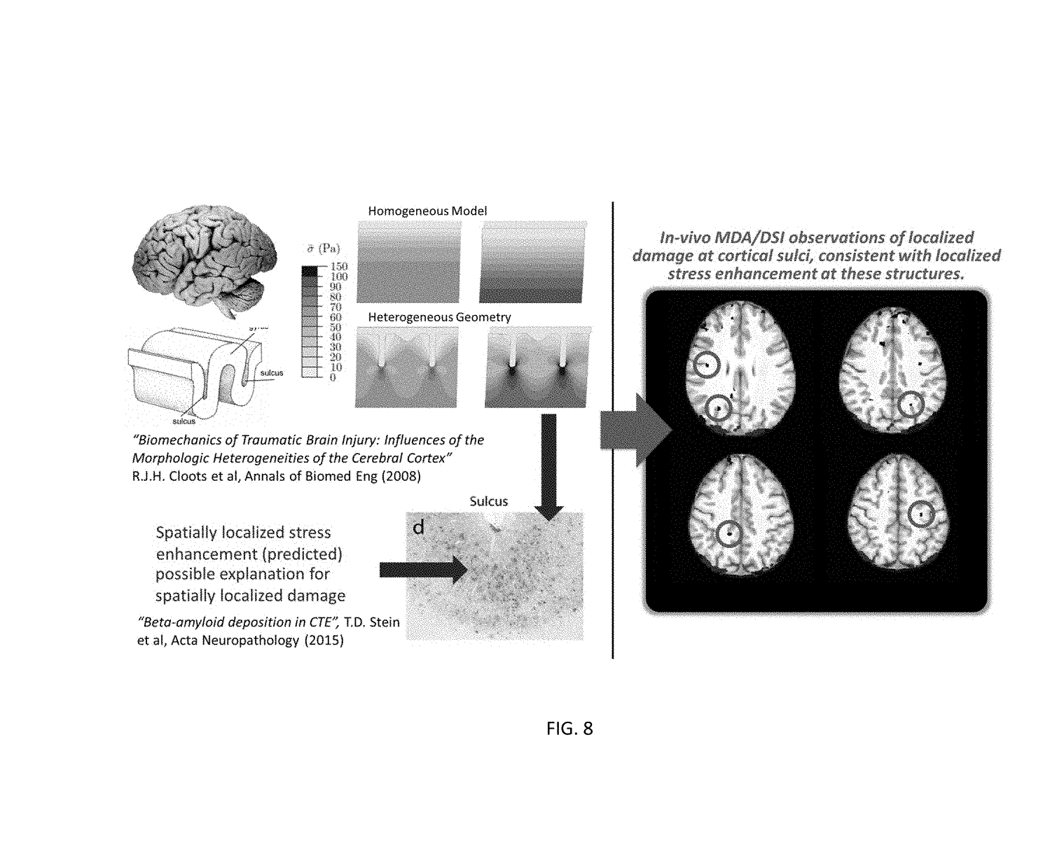

Above the damage threshold illustrated in FIG. 2 and FIG. 5, additional spatially localized damage is also observed in-vivo at the cortical sulci, consistent with localized stress enhancement at these structures, as shown in FIG. 8. Finite element brain models that include tissue specific mechanical properties and detailed structural morphologies have predicted several important impact responses, including spatial localization of stress fields and tissue damage at morphologic features such as the cortical sulci. One implication of this finding is that even modest impacts, at levels traditionally thought to be safe, may generate localized regions of high stress and damage in the brain, which has been proposed as one possible explanation for recent observations that beta amyloid deposition is concentrated at the cortical sulci in the brains of professional football players who were diagnosed post mortem to have suffered from CTE. The ability to measure the onset of such damage much earlier in an athlete's career is critical to understanding of how such effects originate and evolve over time. In this invention, as illustrated in FIG. 8, we have demonstrated for the first time that in-vivo localized damage at the cortical sulci can be observed following head impacts in sports, consistent with the predicted localized stress enhancement at these features, and revealing that the onset of brain injuries and potentially CTE may begin far earlier than previously recognized. Furthermore, as illustrated in FIG. 5, we have developed a corresponding IMD device and neuro-mechanical biomarker method to detect the onset of this damage and alert users and supervisory personal in real time.

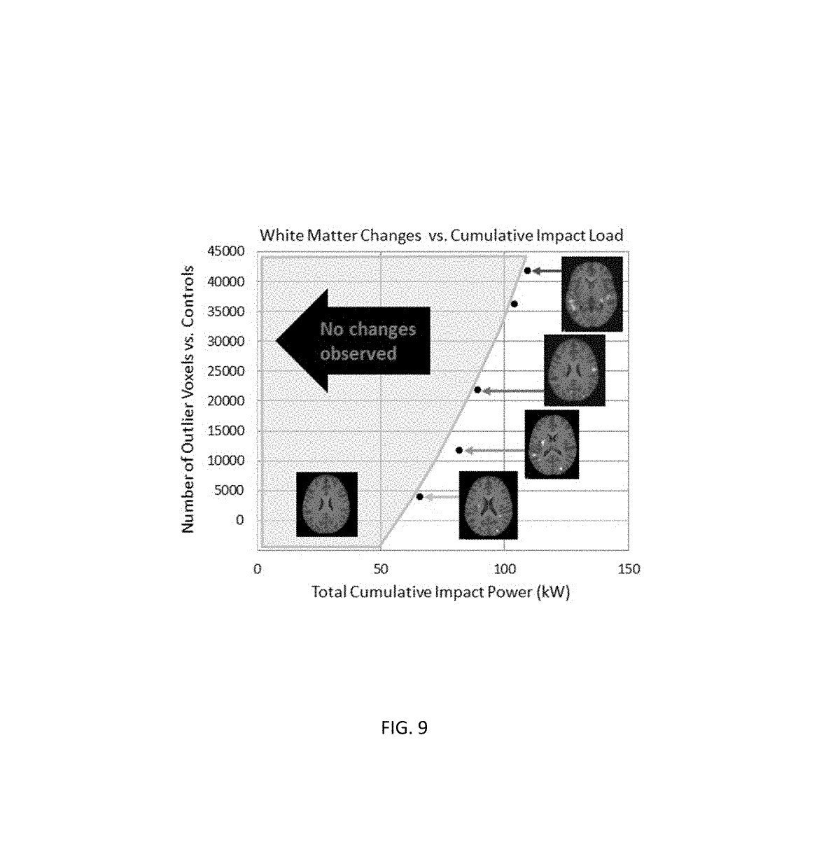

FIG. 9 Illustrates IMD calibration using the number of DSI outlier voxels observed as a function of total cumulative impact power transferred to the brain. Physiological changes, biochemical responses, injuries, and any observed symptoms are all triggered by the mechanical energy transferred to the head/neck/brain during direct or indirect head impacts. Since many of these changes are cumulative, any metric used to characterize the corresponding impact injury risks and potential severity should be directly measurable and cumulative. Earlier studies of automobile collisions and sports impacts showed that the maximum head impact power during a single impact was found to be a good predictor of concussions, and that effective thresholds for 50%/95% probability of a concussion injury due to a single impact were on the order of 13 kW/21 kW, respectively. Impact power transferred to the brain has not been investigated, nor has the application of cumulative impact power to assess cumulative brain trauma due to lower-intensity head impacts.

The present invention demonstrates that the cumulative mechanical power transferred to the brain is a valid neuro-mechanical biomarker for cumulative impact trauma, and that the linear, rotational, and total mechanical power can be calculated directly from the outputs of a MEMS accelerometer and MEMS gyroscope within a universally deployable wearable device. In addition, most spurious impacts, even if they register high peak linear and rotational accelerations, are observed to have small physical displacements, and hence contribute minimal errors to measurements of cumulative impact power.

The observation that global diffusion changes emerge throughout an athletic season as players accumulate head impacts (FIGS. 4 and 6) is consistent with finite element modeling of head impacts, which predict that relative displacements and deformations are widely distributed throughout the brain due to coupling of linear and rotational degrees of freedom.

The present invention might also help to assess reorganization or recovery processes occurring after head trauma, and therefore enable the investigation of white matter plasticity. As shown in FIGS. 4, 6, and 7, sub-concussive head impact trauma at levels typical of a wide range of athletic activities appears to induce both reversible and persistent changes that are detectable using brain imaging techniques.

This invention has shown that the measurement of longitudinal changes in white matter diffusion using high-angular-resolution DSI imaging and voxel-wise MDA estimates, combined with the measurement of head impact biomechanics using wearable sensors, enables detection and characterization of sub-concussive head trauma via calibration of the sensor using the DSI results. The current invention may also be utilized to characterize localized mechanical damage thresholds and the temporal evolution of corresponding physiological, biochemical, and neuropsychological manifestations.

These findings demonstrate the applicability and reliability of DSI techniques for assessing brain injuries at a microscopic level, and reveal the correlation between neuro-mechanical biomarkers and neuroimaging biomarkers in deriving parameters of tissue integrity/physiological changes.

The present invention is generally applicable to any body part susceptible to mechanical impact injuries and to which an IMD can be attached using methods described in further detail below. An IMD attached to a specific body part can be used to determine the above impact forces, torques, displacements, and impact durations, which in turn can be used to calculate the energy and power transferred to the body part due to impact exposure. It is an object of this invention to use biomechanical sensor data to provide critical mechanical loading information for single impact events and cumulative exposure to multiple impacts over time. A biomarker derived directly from the mechanical loading and correlated with physiological changes that are indicative of injury or progressive deterioration to the specific body part as a function of impact biomechanics would have universal applicability for athlete safety and performance monitoring, and may help to better understand the early biological changes which occur after acute injury.

One or more components of the transferred power can be used as a biomarker which, when compared to physiological change thresholds, is indicative of the potential severity of the injury resulting from the impact exposure. Physiological change thresholds can be stored in the IMD for a population of similar users, and be used to alert the wearer or supervisory personnel in real time when a specific injury threshold is being approached or has been exceeded.

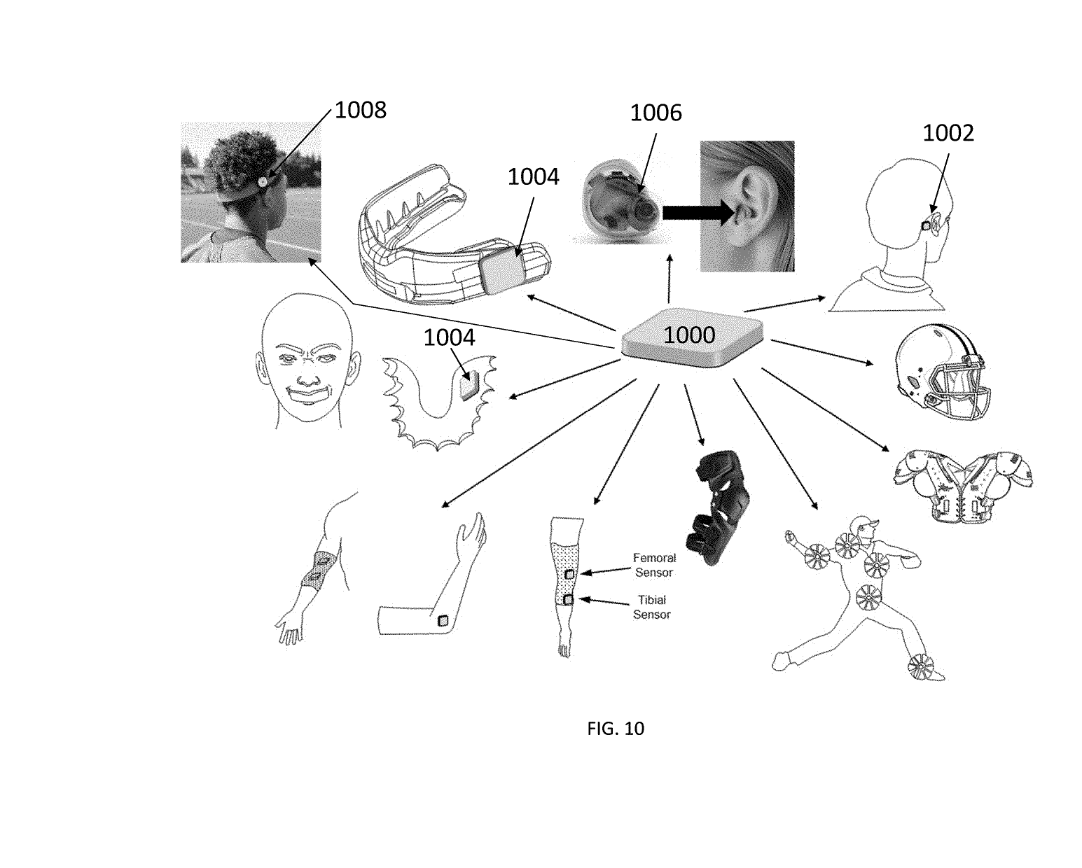

As illustrated in FIG. 10, in a preferred embodiment of the invention the IMD 1000 may be attached to the skin at the mastoid 1002 of the user, inserted into a retaining device fitted to the upper teeth or palate 1004 of the user, placed within an ear piece shaped enclosure 1006 that can be inserted into the user's auditory canal to provide efficient and low-distortion mechanical coupling to the head, or attached to a headband 1008 worn by the user.

As illustrated in FIG. 10, in another embodiment of the invention, the IMD may be worn on the elbow, knee, ankle, or other parts of the body susceptible to injuries due to mechanical impacts. In accordance with this invention, a single IMD may be used, or two or more IMDs may be used simultaneously, examples being one device behind each ear or one device on either side of the knee or elbow joint, as illustrated in FIG. 10. In other embodiments of the invention, the IMD may be inserted into a slot or pouch in a sleeve, shirt, shorts, leggings, other garment, belt, glove, shoe, or other item worn by the user for the purpose of monitoring impact injury risks. In other embodiments of the invention, the IMD may be attached to or incorporated into protection gear, including but not limited to helmets, shoulder pads, elbow pads, protective gloves, thigh pads, knee pads, angle pads, and footwear.

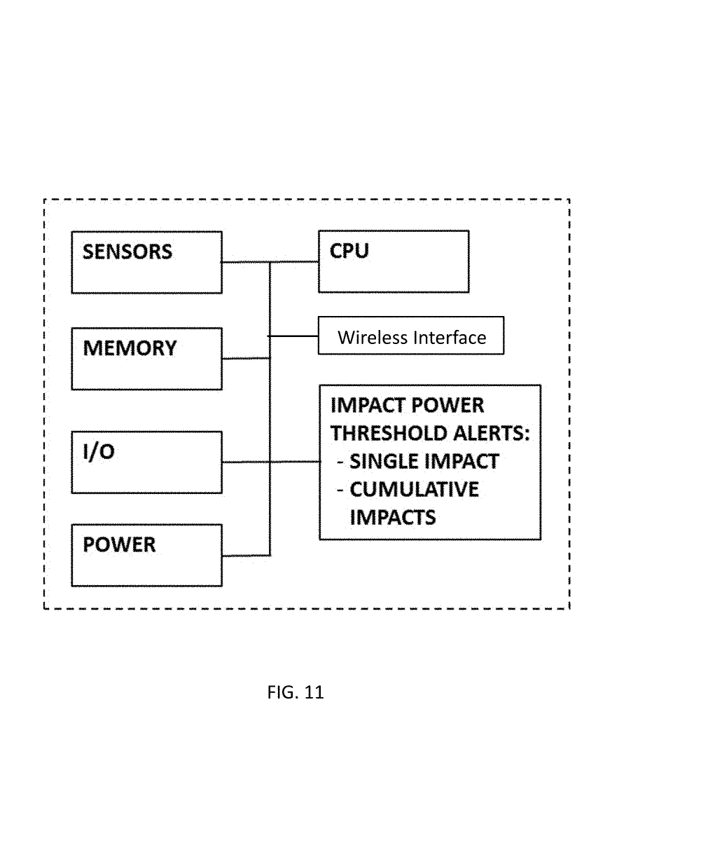

With reference to the block diagram of FIG. 11, in a preferred embodiment of the current invention the IMD includes at least one sensor, a central processing unit (CPU), memory, I/0 means for input/output of control signals and data to/from the device, one or more audio, vibrational, or optical means to present neuro-mechanical biomarker threshold alerts, a wireless interface for communication with another computing device, such as an application running on a mobile device or a device-monitoring hub, router, or server, and a battery or other means of supplying power. The CPU includes a processor, data memory to receive and store data from the linear and rotational motion sensors, and instruction memory to store programming instructions operable by the processor to perform a variety of functions as desired, including calculation of the neuro-mechanical biomarker.

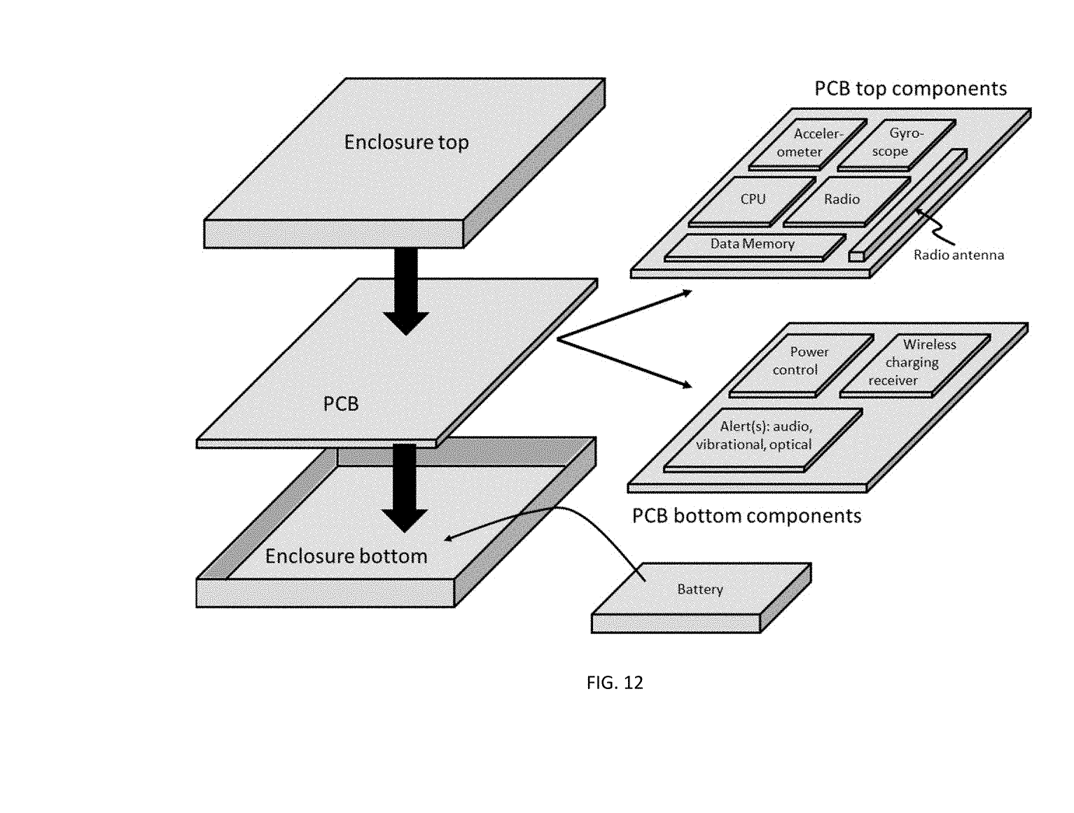

In accordance with the preferred embodiment of the invention shown in FIG. 12, the IMD is configured with an accelerometer able to detect acceleration along three linear axes, and a gyroscope able to detect angular rate of rotation about three axes. In one embodiment, both the accelerometer and the gyroscope are microelectromechanical sensors (MEMS) devices that utilize precise measurement of the deflection of miniature weighted cantilever beams, membranes, or spring assemblies to accurately measure linear acceleration or angular velocity experienced by the sensor. In one embodiment, the minimum sampling rates of both the accelerometer and the gyroscope are 500 kHz to accurately capture the head impact response relevant to injury.

As illustrated in FIG. 12, the IMD includes an electronics module in which the sensors, CPU, memory, and other electronics components described in FIG. 11 are mounted on a printed circuit board (PCB), which also provides electrical connectivity between the various components. The PCB may incorporate one or more layers of contact traces, and may be assembled from a hard or a flexible material. The PCB and any additional device components are preferably enclosed or embedded within an impact resistant and hermetically sealed protective enclosure to protect the entire assembly from damage due to mechanical contact or exposure to moisture, perspiration, or other potentially damaging environmental elements.

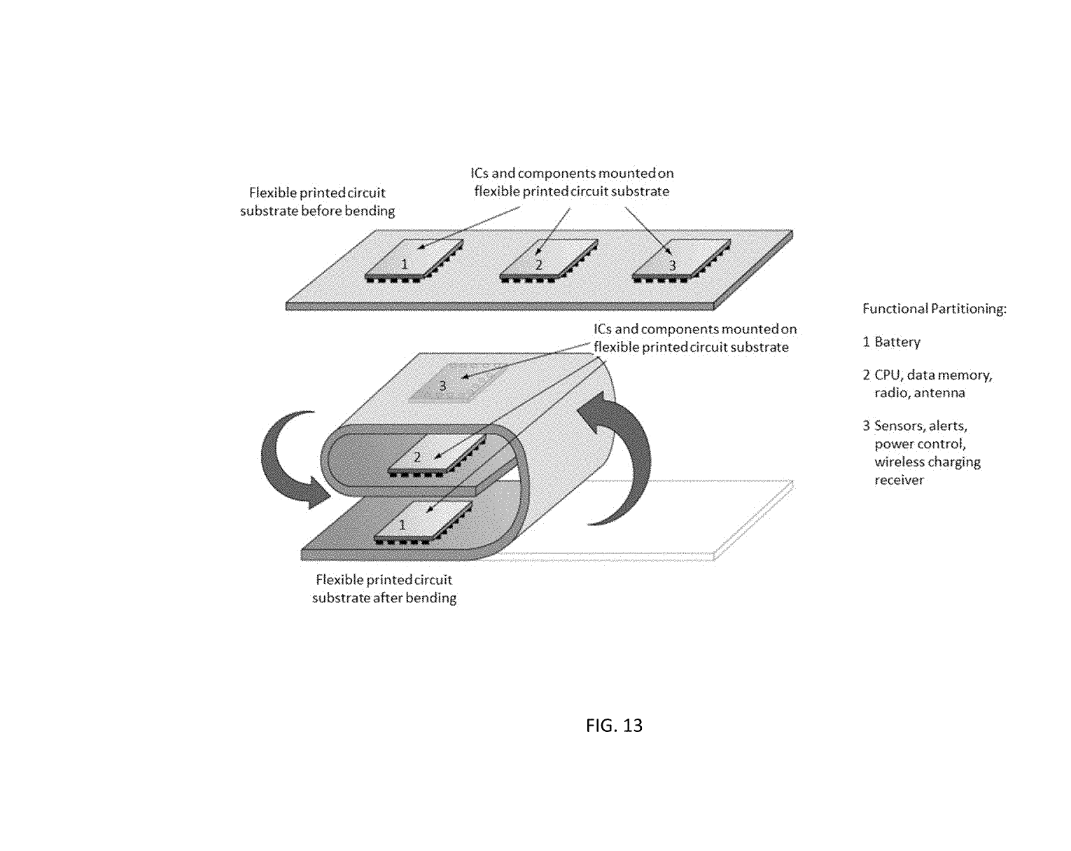

As illustrated in FIG. 13, further miniaturization of the IMD may be achieved by using a folded flexible printed circuit substrate. One or more ICs and other components may be integrated together on rigid or flexible PCBs to form functional sub-modules. These sub-modules are then mounted onto the flexible printed circuit substrate prior to folding. The flexible printed circuit substrate provides electrical connectivity between the sub-modules. FIG. 13 shows a preferred embodiment of the invention, in which the various functions of the IMD are partitioned into three sub-modules.

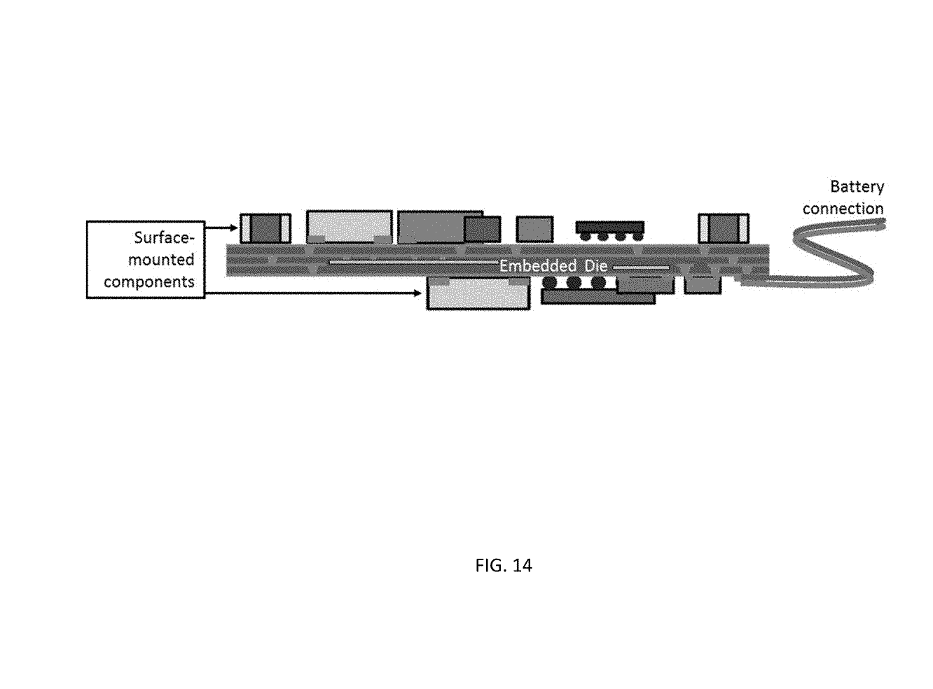

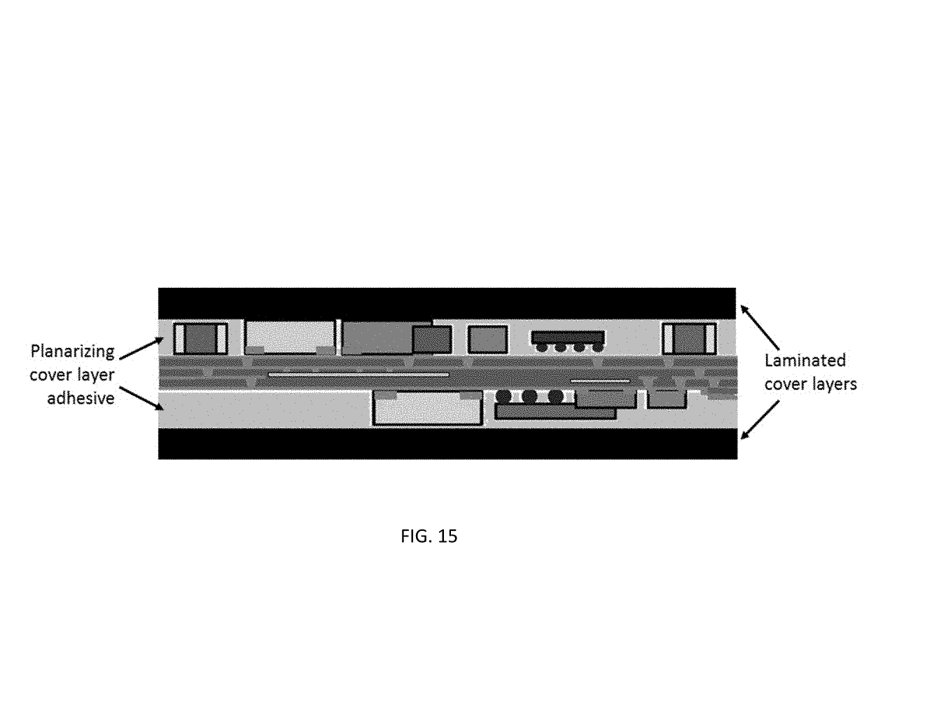

As illustrated in FIG. 14, the thickness of the IMD may be reduced by embedding one or more components as bare die within a flexible circuit substrate. As illustrated in FIG. 15, the thickness of the IMD may be reduced by using laminated cover layers and planarizing cover layer adhesive layers.

To fit behind ear, the overall dimensions of the IMD should not exceed approximately 22 mm in length, 13 mm in width, or 5 mm in thickness. To avoid distortions in motion measurement due to mass-spring motion of the sensor on the user's skin, the mass of the IMD should not exceed 2 gr. To eliminate external electrical contacts and enable hermetic sealing for environmental stability and in-mouth applications, the IMD utilizes wireless input and output of control and data signals, as well as wireless power delivery for battery charging and operation. To enable secure and simultaneous data and control I/O to 10 or more devices over a range of 100 m or greater (typical of an athletic playing field or stadium), and connectivity to existing smart grid or smart city utility networks, the IMD utilizes a multi-channel 868/915 MHz radio transceiver.

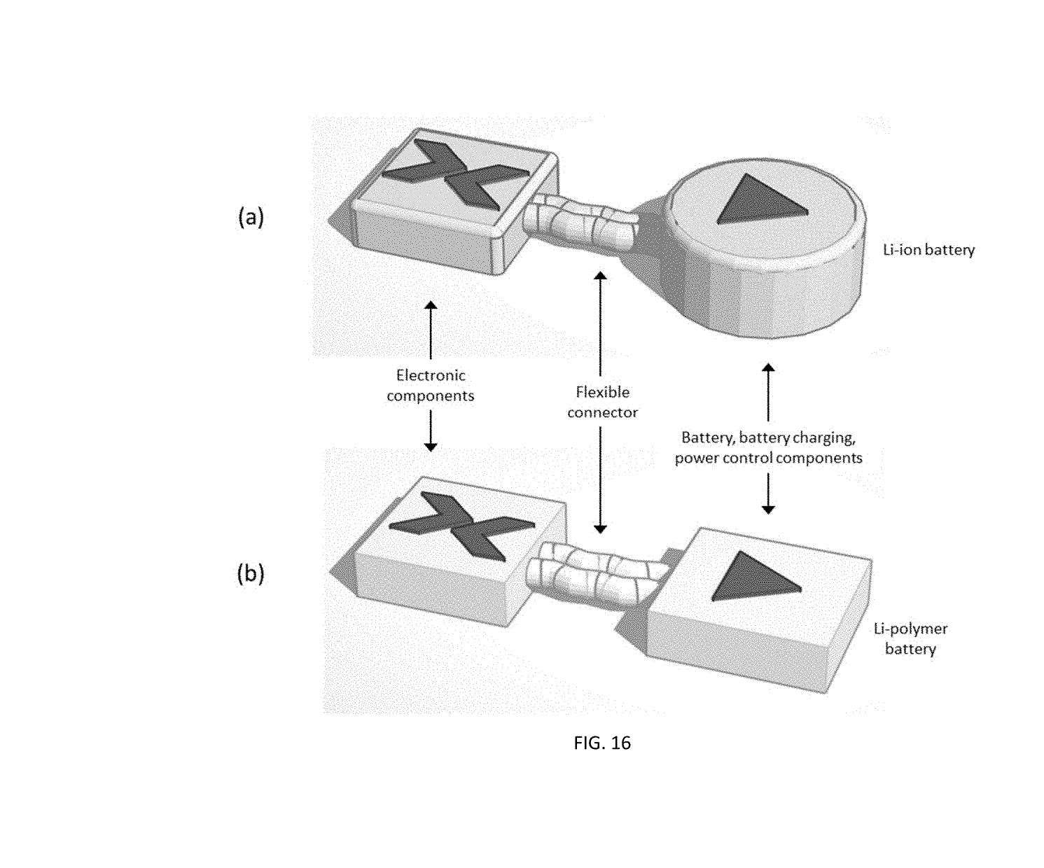

Since the single heaviest component in the IMD is typically the battery, the mass of the impact sensor can be further reduced, to minimize motion on the user's skin, by segmenting the device into two sections as illustrated in FIG. 16. One section contains only those electronic components required for calculation of the neuro-mechanical biomarker, while the other section contains the battery. Depending on battery capacity and mechanical resilience requirements, the battery may be either a cylindrical Li-ion battery, or coin cell, as shown in FIG. 16(a), or a rectangular Li-polymer battery, as shown in FIG. 16(b). A flexible connector links the two sections, as shown in FIG. 16, and separate adhesives are used to attach each section to the user's skin to mechanically decouple the lighter sensor section from the heavier battery section.

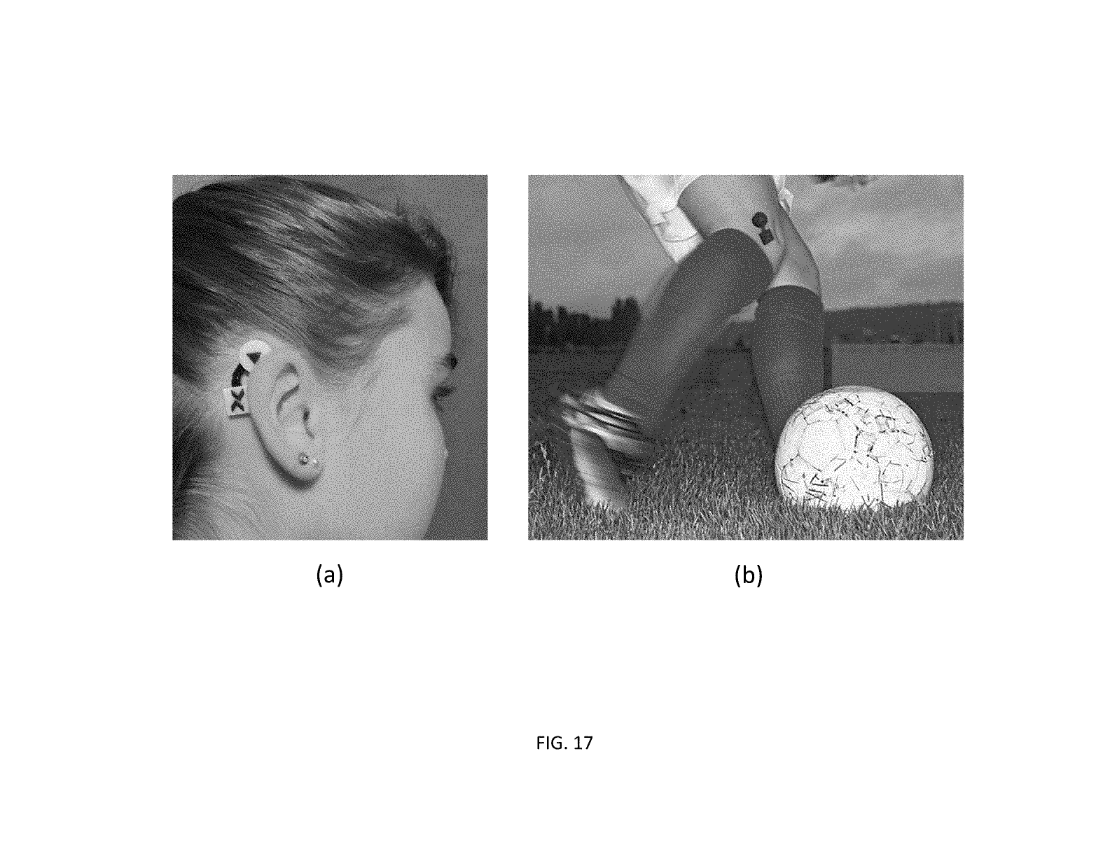

FIG. 17 shows illustrates a two-section IMD worn on (a) the user's head, and (b) the user's knee. As shown in FIG. 17(a), an additional benefit of the two-section sensor is that it enables greater flexibility in placement behind the user's ear.

In one version of the invention, a separate adhesive sticker is used to attach the IMD to the user. In this configuration, the sticker may be discarded after each use. In a preferred version incorporating a separate adhesive sticker, the sticker includes a device adhesion area that matches the footprint of the IMD surface to which the sticker is to be attached. The sticker includes a backing sheet that covers the IMD adhesive area until the sticker and IMD are ready for use. At the time of use, the backing sheet is removed to expose the adhesive and the IMD is attached to the exposed adhesive of the device adhesion area. The opposing side of the sticker likewise includes a backing sheet covering an adhesive formulated to stick to the user's skin. To attach the IMD to the user's head, such as shown in FIGS. 10 and 17, the backing sheet is removed and the back side of the sticker is attached to the skin over the mastoid process behind the user's ear. This configuration likewise attaches the IMD to the wearer, as shown in FIGS. 10 and 17, because the IMB is adhered to the sticker, which in turn is adhered to the skin. Thus, an embodiment of the invention is a kit comprising the IMB and a set of adhesive stickers.

In an alternate version of the invention, the sticker includes an adhesive back side as described above to attach to the user, but incorporates a hook and loop (“Velcro”) or similar fastener on the front side for attaching the IMB to the sticker; the front side of the sticker includes a first component of a hook and loop fastener while the back side of the IMB includes the second complementary component of a hook and loop fastener, thereby allowing the IMD to be removably attached to the sticker.

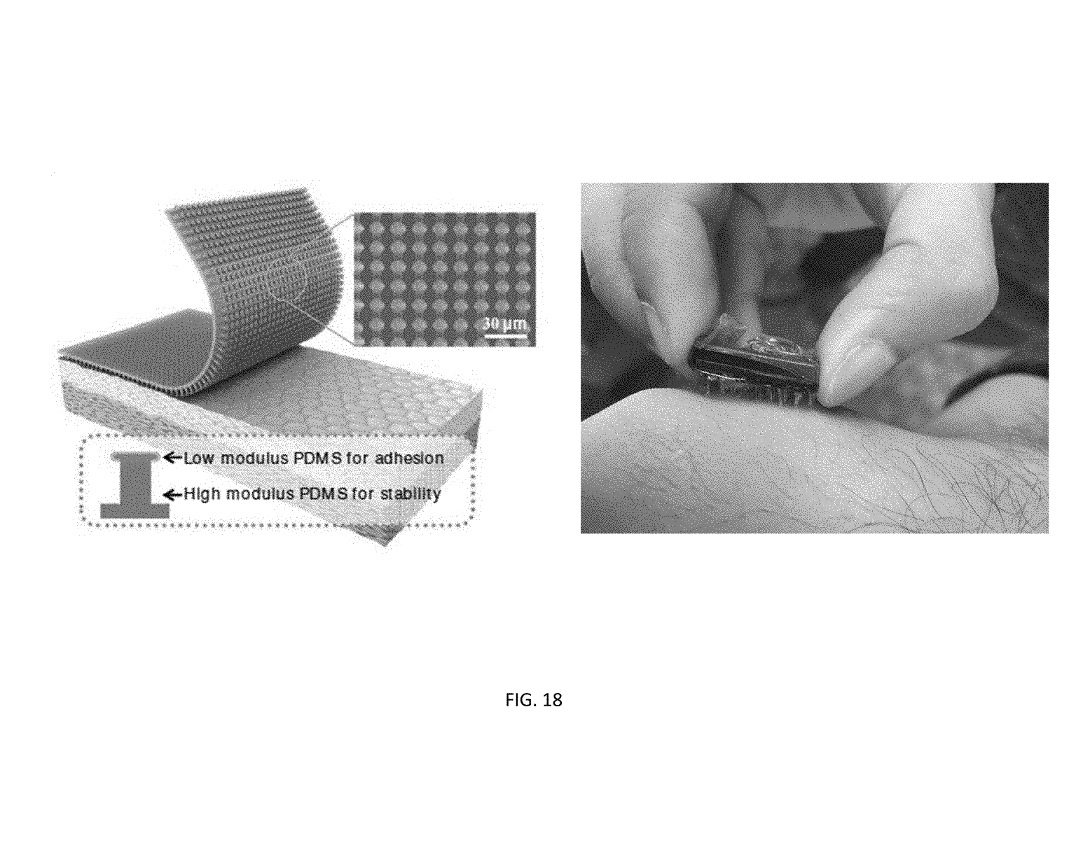

In another alternative version of the invention, a reusable adhesive is attached to the skin-facing side of the IMB, as shown in FIG. 18. The reusable adhesive may be applied to the IMB at the time of manufacture, or may be applied in a separate step prior to first use. In one embodiment of the invention, the reusable adhesive consists of an array of micro-structured polydimethylsiloxane (PDMS) pillars. As shown in FIG. 18, a high modulus PDMS material is used to provide a stable base for each of the pillars attached to the IMB. Suction-cup shaped mushroom caps are structured on top of the pillars from a low modulus PDMS material to provide good adhesion to the skin. The angle of the pillars is designed to allow the IMD to be removed by tilting in one direction with respect to the skin, such as rotating the device away from the user's ear.

In further alternate versions of the invention, the sticker includes a sleeve, box, pouch, or other similar enclosure into which the IMB can be removably inserted.

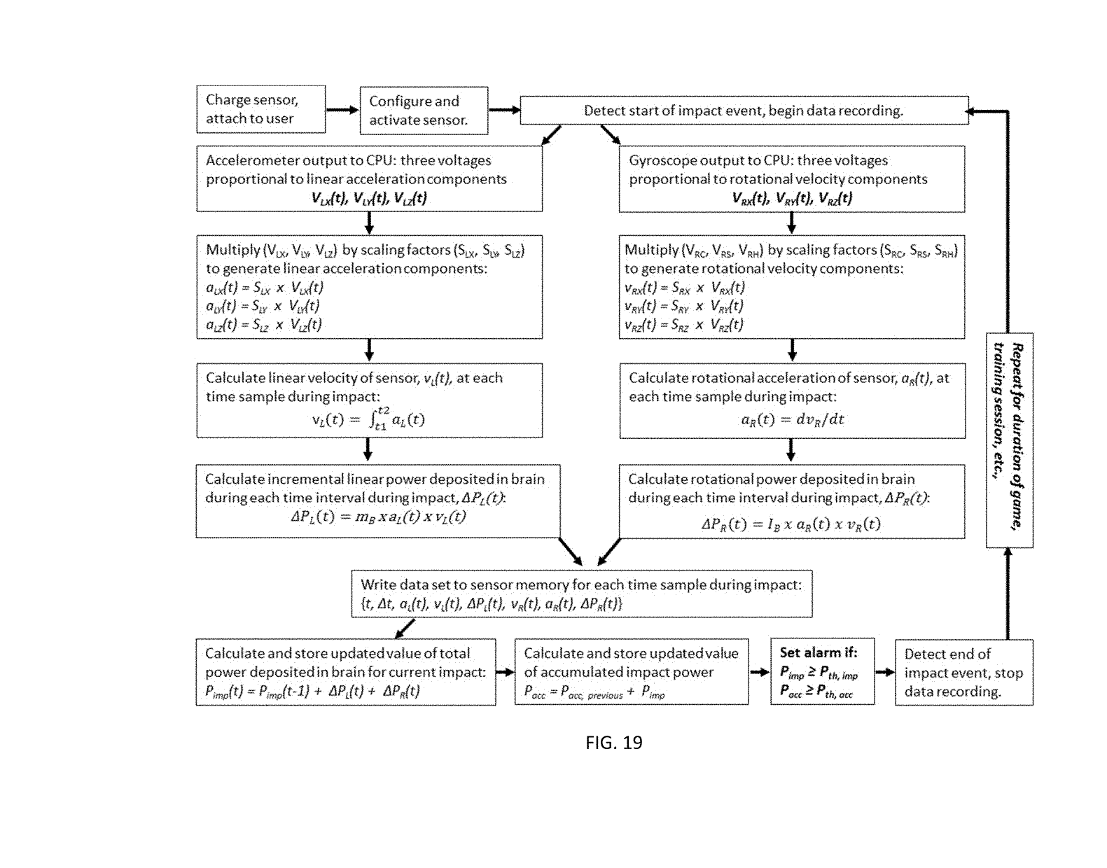

FIG. 19 illustrates device deployment and data flow for a preferred embodiment of the current invention, in which the IMB calculates the impact power Pᵢₘₚ transferred to the brain from an individual impact; updates the accumulated impact power P_{acc} transferred to the brain for all impact events that have occurred during the current period of time for which this value is being monitored; utilizes one or more of the twelve individual x, y, z components of the linear and rotational contributions to Pᵢₘₚ and P_{acc}, or a combination of any two or more components, to calculate the value of a neuro-mechanical biomarker Bₙₘ; compares the calculated value of Bₙₘ to a corresponding injury risk threshold value stored in the device for the wearer; and sets an alarm if a threshold is met or exceeded. In the preferred embodiment, all of the above steps are implemented substantially in real time at the time immediately following each impact event to which the user is exposed.

MEMS accelerometers measure the linear components of an applied force. The force of an impact generates a displacement of an internal test mass mounted on a miniature cantilever beam, membrane, or spring assembly, which changes the internal capacitance of the structure and generates a voltage. A three axis MEMS accelerometer generates three output voltages V_{LX}(t), V_{LY}(t), T_{LZ}(t) that are proportional to the three corresponding linear components of the applied force due to the impact. These output voltages are read by the processor. Given the calibrated relationship between the applied force, the internal test mass, and the resulting acceleration of the sensor, calibrated scaling factors S_{LX}, S_{LY}, S_{LZ} are generated for the accelerometer and provided by the manufacturer. The three linear acceleration components of the sensor's motion are calculated in the IMD CPU by multiplying the measured output voltages by the corresponding scaling factors:

a_{LX}(t)=S_{LX}×V_{LX}(t)

a_{LY}(t)=S_{LY}×V_{LY}(t)

a_{LZ}(t)=S_{LZ}×V_{LZ}(t)

MEMS gyroscopes measure the angular components of any rotational velocity of the head and sensor that results from an impact. The rotational motion also generates a displacement of an internal test mass mounted on a miniature cantilever beam, membrane, or spring assembly, which changes the internal capacitance of the structure and generates a voltage. A three axis MEMS gyroscope generates three output voltages V_{RX}(t), V_{RY}(t), V_{RZ}(t) that are proportional to the three corresponding rotational velocity components generated by the impact. These output voltages are read by the processor. Given the calibrated relationship between the applied torque, the moment of inertia of the internal test mass, and the resulting rotational velocity of the sensor, calibrated scaling factors S_{RX}, S_{RY}, S_{RZ} are generated for the gyroscope and provided by the manufacturer. The three rotational velocity components of the sensor's motion are calculated in the IMD CPU by multiplying the measured output voltages by the corresponding scaling factors:

V_{RX}(t)=S_{RX}×V_{RY}(t)

V_{RY}(t)=S_{RY}×V_{RY}(t)

V_{RZ}(t)=S_{RZ}×V_{RZ}(t)

With efficient and low-distortion coupling between the sensor and the head, as provided for in the invention, the linear and rotational motion of the head at the location of the sensor will be the same as the linear and rotational motion of the sensor itself.

With reference to FIG. 19, once the IMD has detected the start of an impact event, the processor begins reading the accelerometer output voltages V_{LX}(t), V_{LY}(t), V_{LZ}(t) at a pre-selected sampling rate for the duration of the impact event, and calculates the sensor linear acceleration components a_{LX}(t), a_{LY}(t), a_{LZ}(t) as described above for each sampling time interval Δt during the impact event. Assuming that the linear motion of the brain is the same as the linear motion of the head, the corresponding incremental linear power ΔP_{L}(t) transferred to the brain during each time interval Δt during an impact event can be calculated in the processor:

$$Δ {P}_{L} ( t ) = \frac{{m}_{B} \times {a}_{L} ( t ) \times Δ {d}_{L} ( t )}{Δ t}$$

where m_{B} is the mass of the brain, a_{L}(t) is the linear acceleration during the time interval Δt, Δd_{L}(t) is the incremental linear displacement of the sensor during the time interval Δt, v_{L}(t)=Δd_{L}(t)/Δt is the linear velocity during the time interval Δt beginning at t₁ and ending at t₂, calculated as

v_{L}(t)=∫ₜ₁^{t2}a_{L}(t)

The processor also reads the gyroscope output voltages V_{RX}(t), V_{RY}(t), V_{RZ}(t) at a pre-selected sampling rate for the duration of the impact event, and calculates the sensor rotational velocity components v_{RX}(t), v_{RY}(t), v_{RZ}(t) as described above for each sampling time interval Δt during the impact event. Assuming that the rotational motion of the brain is the same as the rotational motion of the head, the corresponding incremental rotational power ΔP_{R}(t) transferred to the brain during each time interval Δt during an impact event can be calculated in the processor:

$$Δ {P}_{R} ( t ) = \frac{{I}_{B} \times {a}_{R} ( t ) \times Δ {θ}_{R} ( t )}{Δ t}$$

where I_{B} is the moment of inertia of the brain, Δ_{θ}_{_{R}}(t) is the incremental angular rotation of the sensor during the time interval Δt, v_{R}(t)=Δ_{θ}_{_{R}}(t)/At is the angular velocity during the time interval Δt, and a_{R}(t) is the angular acceleration of the sensor during the time interval Δt beginning at t₁ and ending at t₂, calculated as

a_{R}(t)=dv_{R}/dt

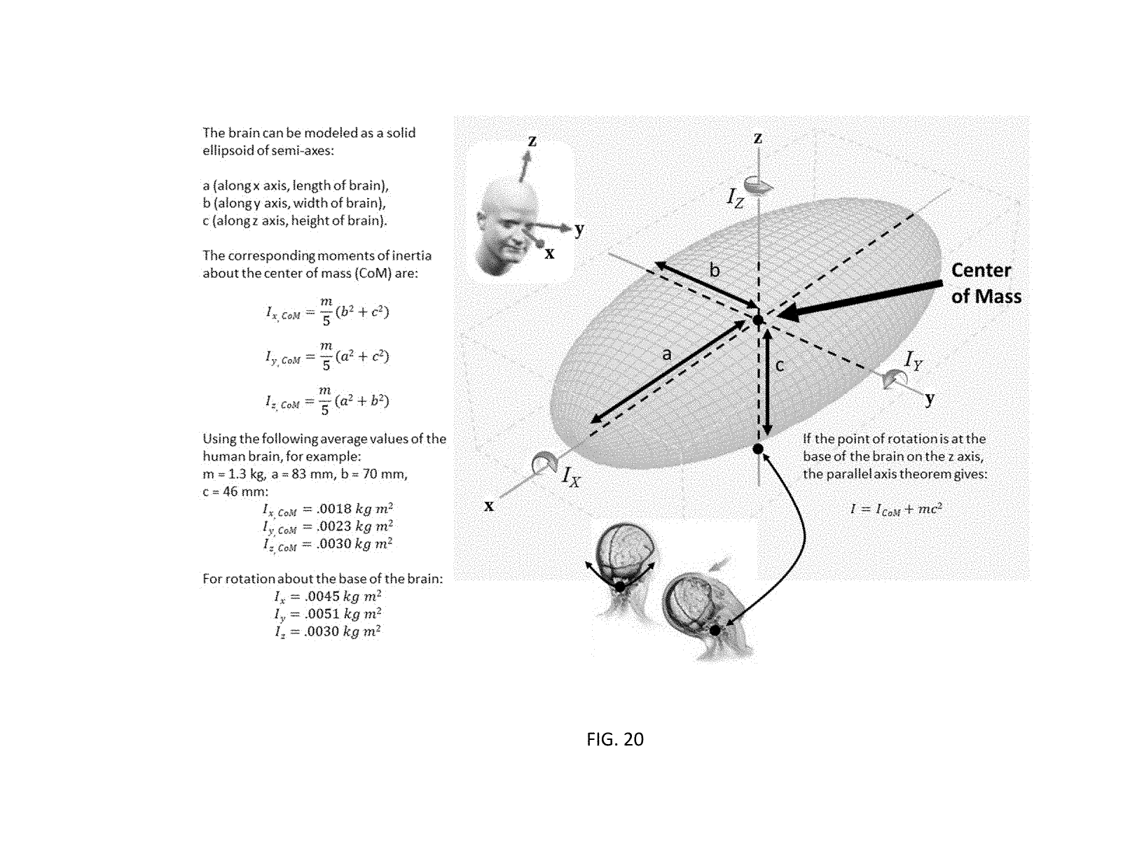

As shown in FIG. 20, the moments of inertia of the brain can be calculated by modelling the brain as a solid ellipsoid of semi-axes a (along x axis, length of brain), b (along y axis, width of brain), and c (along z axis, height of brain). The corresponding moments of inertia about the center of mass (CoM) of the brain are then given as:

$${I}_{x , CoM} = \frac{m}{5} ( {b}^{2} + {c}^{2} )$$

$${I}_{y , CoM} = \frac{m}{5} ( {a}^{2} + {c}^{2} )$$

$${I}_{z , CoM} = \frac{m}{5} ( {a}^{2} + {b}^{2} )$$

Using the following average values of the human brain, for example: m=1.3 kg, a=83 mm, b=70 mm, c=46 mm:

I_{x,CoM}=0.0018 kg m²

I_{x,CoM}=0.0023 kg m²

I_{x,CoM}=0.0030 kg m²

As shown in FIG. 20, the head and brain are not free to rotate in an unconstrained manner, but instead are constrained by the neck and spine to rotate about a fixed point at the base of the brain on the z axis. For rotation about the base of the brain, the parallel axis theorem gives:

I=I_{CoM}+mc²

The corresponding moments of inertia for the brain are then given by:

Iₓ=0.0045 kg m²

I_{y}=0.0051 kg m²

I_{z}=0.0030 kg m²

The above values for the moment of inertia of the brain are consistent with those measured using human cadavers and calculated using detailed finite element models of the human head, neck, and brain.

At the end of each time interval Δt during an impact event, the CPU stores the following data in the IMD's data memory:

{t₁, t₂, Δt, a_{L}(t), ΔP_{L}(t), v_{R}(t), a_{R}(t), ΔP_{R}(t)}

The processor also calculates and stores updated values of the total impact power transferred to the brain for the current impact

Pᵢₘₚ(t)=Pᵢₘₚ(t−1)+ΔP_{L}(t)+ΔP_{R}(t)

At the end of each impact event, the CPU calculates and stores the final value of the total power transferred to the brain during the impact event, Pᵢₘₚ. The CPU then calculates and stores an updated value of the accumulated impact power for all impact events that have occurred during the current period of time for which this value is being monitored:

P_{acc}=P_{acc,previous}+Pᵢₘₚ

The CPU then utilizes one or more of the twelve individual x, y, z components of the linear and rotational contributions to Pᵢₘₚ and P_{acc}, or a combination of any two or more components, to calculate the value of a neuro-mechanical biomarker Bₙₘ. Next, the CPU compares the calculated value of Bₙₘ to a corresponding injury risk threshold value stored in the device for the wearer; and sets an alarm if a threshold is met or exceeded. For example, the CPU may calculate Bₙₘ=Pᵢₘₚ and set an alarm if Pᵢₘₚ exceeds a single impact power threshold value that has been stored for the device wearer (Pᵢₘₚ≥P_{th,imp}) or if P_{acc} exceeds an accumulated impact power threshold value that has been stored for the device wearer (P_{acc}≥P_{th,acc}).

Once an alarm has been set as described above, the IMD can utilize one of several methods to signal the alarm condition directly to the wearer of the device or to supervisory personnel monitoring the wearer of the device. In some embodiments of the invention, the I/O component of the IMD may include the ability to signal the alarm condition directly to the wearer through an onboard vibrational or auditory element, such as a MEMS speaker, or directly to local supervisory personnel via an auditory element, such as a MEMS speaker, or an optical element, such as an LED. In other embodiments, the alarm signal is transmitted from the IMD to the charger/wireless base station using the long range 868/915 MHz radio, and may be routed further from the charger/wireless base station to supervisory personnel devices or other remote locations via Bluetooth, Wi-Fi, or USB connectivity.

In the preferred embodiment, all of the above steps are implemented substantially in real time at the time immediately following each impact event to which the user is exposed.

A key benefit of the invention compared to other approaches for impact injury monitoring is the elimination of the need to carry out iterative manual inspections and computerized analyses of sensor motion traces to filter out non-impact events. Even if these non-impact events generate spurious motion of the IMD with large peak linear or rotational accelerations, the linear displacement of the sensor during these spurious impacts (˜1 mm) is typically at least two orders of magnitude lower than during a real impact (˜10 cm), hence the velocity and resulting contribution to the measured power is typically negligible. In one embodiment of the present invention, spurious impacts can be filtered from the measured power by neglecting any events for which the linear displacement of the sensor is less than a preset threshold, for example 5 mm.

In a preferred embodiment of the current invention, the threshold value Bₙₘ for a single impact is P_{th,imp}, and is initially set equal to 10 kW, and the threshold value of Bₙₘ for cumulative impacts is P_{th,acc}, and is initially set equal to 35 kW within any two-hour period, based on data available from studies completed to date. These injury thresholds can then be adjusted for individual users based on additional data collected for a generalized population of similar users based on metrics such as the user's height, weight, age, gender, strength of one or more portions of their body, previous injury history, and genetic predisposition to injury risk, as well as the specific risk-generating activities in which the user is participating. The injury thresholds may also include dynamic metrics such as the user's level of hydration, fatigue level, recent energy expenditure, and levels of specific electrolytes, metabolites, brain injury biomarkers, or other chemical substances measured in the user's cerebrospinal fluid, synovial fluid, blood, saliva, perspiration, or urine.

In a preferred embodiment of the invention, a lookup table is stored in the onboard IMD memory, and contains threshold values of Bₙₘ and values of all static and dynamic metrics used to calculate Bₙₘ for the user. The lookup table may be updated manually during IMD configuration, or may be updated automatically as part of a computer or cloud-based injury management application or service.

In another preferred embodiment of the invention, more complex shapes than the solid ellipsoid illustrated in FIG. 20 may be used as a refinement to the calculations of the moments of inertia and rotational axes of the brain, and these shapes may also include the internal morphological structure and tissue specific mechanical properties of the brain. Differences in the relative motion of the brain with respect to the head and the IMD may also be included as a refinement to the calculation of power transferred to the brain during an impact.

Other embodiments of the current invention may utilize different methods to detect the start and the end of an impact event. The start of an impact event can be based on methods as simple as waiting for the linear acceleration to exceed 10 g, since this is above the maximum value typically observed for simple running motion. Both the start and the end of an impact event can also be determined using more complex motion classification algorithms, including such algorithms available in commercial MEMS sensors.

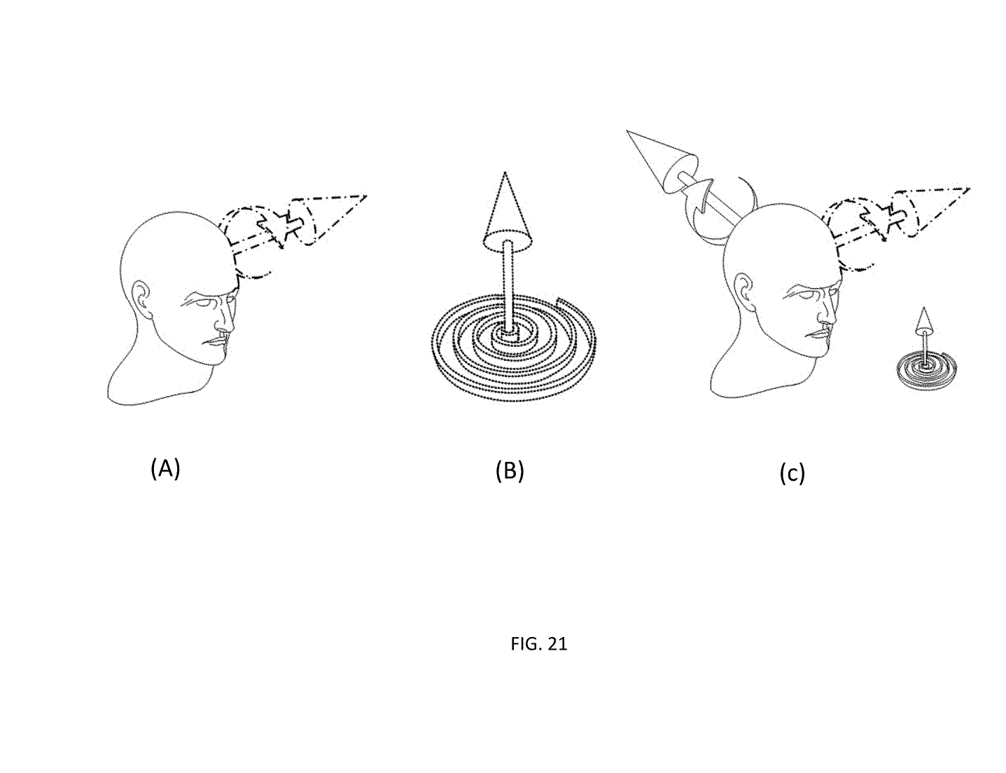

FIG. 21 Illustrates a graphical representation of the neuro-mechanical biomarker Bₙₘ in an embodiment of the current invention. FIG. 21(a) combines a straight arrow and a spiral arrow for each single impact to present the status of the linear and rotational components of Bₙₘ separately. FIG. 21(b) combines a straight arrow and a spiral arrow for cumulative impacts over an extended period of time to present the status of the linear and rotational components of Bₙₘ separately. FIG. 21(c) presents the status of Bₙₘ for both single recent impacts and cumulative impacts in a combined view. As shown in FIG. 21, the graphical presentation of the neuro-mechanical biomarker Bₙₘ can utilize different arrow colors, diameters, or other display parameters to indicate different status levels. For example, values of Bₙₘ well below impact injury thresholds are represented by arrows that are green or have the smallest diameter; values of Bₙₘ within predefined percentages of impact injury thresholds are represented by arrows that are orange or have an intermediate diameter; values of Bₙₘ that exceed impact injury thresholds are represented by arrows that are red or have the largest diameter. In various embodiments of the invention the arrows may be graphically displayed with their origins at the location of the IMD, as is measured, or with their origins at the center of mass of the head, for ease of visual interpretation.

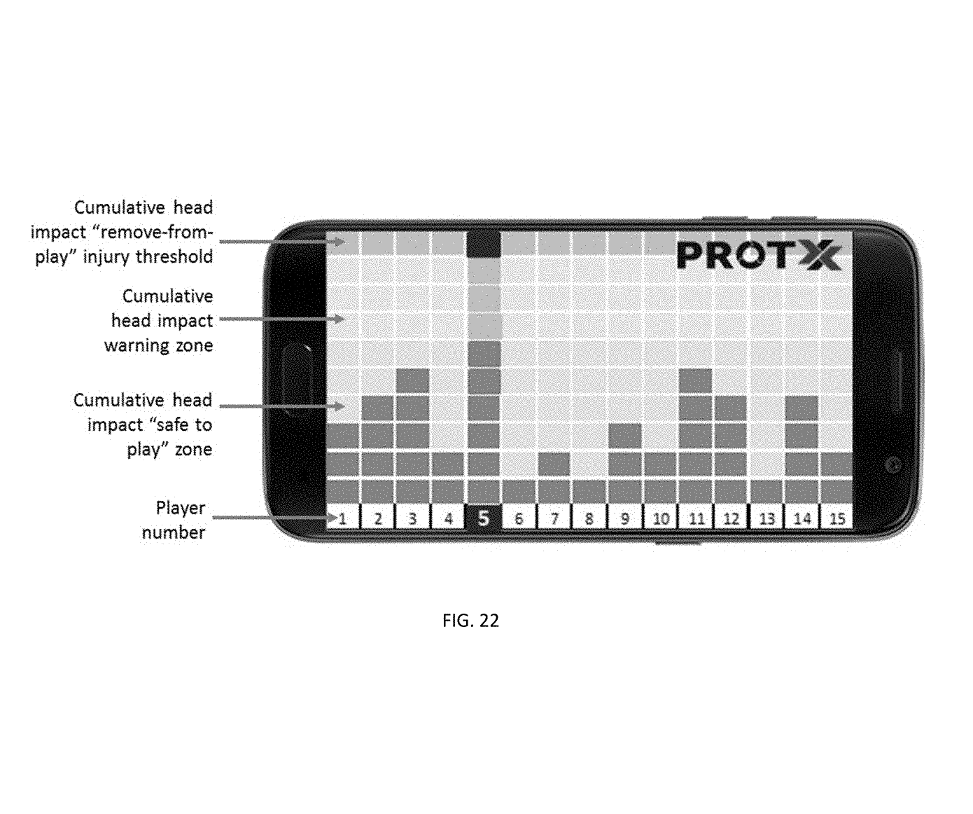

FIG. 22 illustrates the graphical dosimeter presentation on a mobile device, in one embodiment of the present invention, of cumulative head impact power status for a team of athletes or other users.

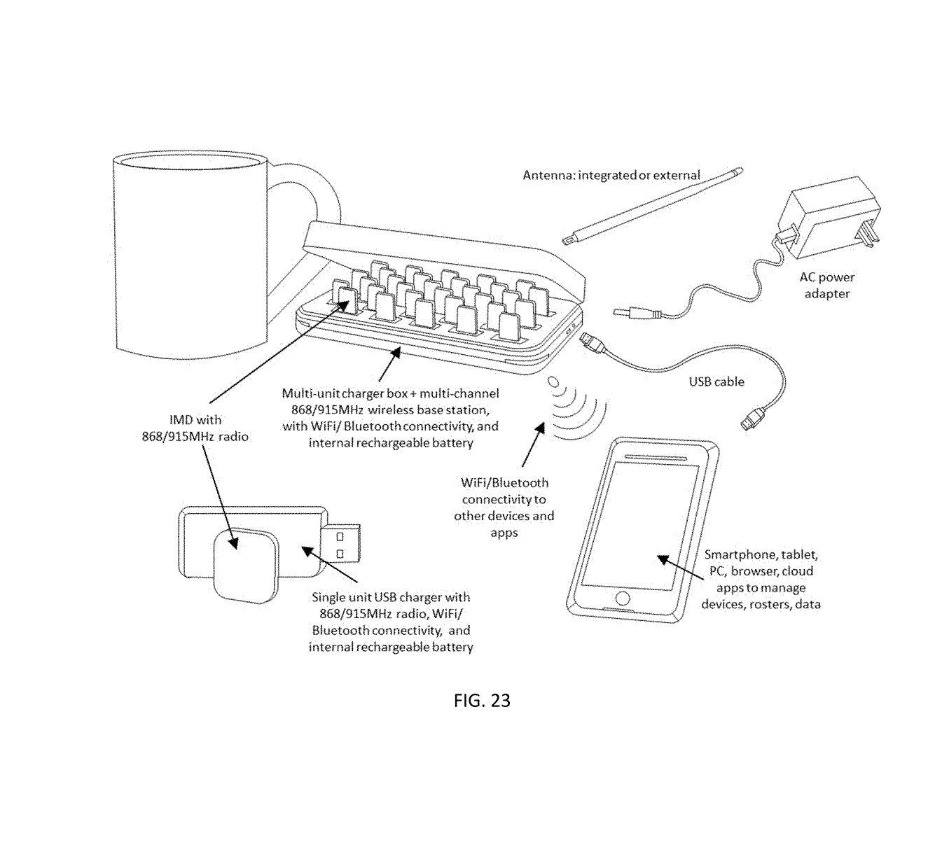

FIG. 23 shows an embodiment of the IMD used in conjunction with additional system components. A multi-unit charger box containing a rechargeable battery is provided in order to store, transport, and charge multiple devices simultaneously. The multi-unit charger box also contains a multi-channel radio transceiver so that it can serve as a wireless base station that communicates with multiple IMD devices simultaneously. In one embodiment of the invention, this wireless communications capability is implemented using multi-channel radio transceiver integrated circuits (ICs), in both the impact sensor device and in the charger box, capable of functioning in the 858 to 928 MHz ISM bands, so that the system is suitable for both European or North American operations, with simultaneous links to tens of devices at a range of 100 m or greater (typical of an athletic playing field or stadium). A dual band ISM radio IC and dual band surface mount chip antenna with a frequency range of 858 to 928 MHz are integrated into the IMD sensor, as shown in FIG. 12. A dual band ISM radio IC and larger format, higher gain dual band 858 to 928 MHz antenna are integrated into the charger box, as shown in FIG. 23.

The multi-unit charger box is further equipped with USB, Wi-Fi, or Bluetooth connectivity in order to communicate with software or browser applications running on smartphones, tablets, other mobile devices, PCs, remote hubs, routers, or servers, or in the cloud, and used to control, configure, or manage the IMD devices. The IMD devices themselves, whether physically inserted in a charger or worn by users, can communicate with the charger box via the multi-channel 868/915 MHz radio link. Functions provided by the software or browser applications include: managing user and team rosters; assigning sensor devices to specific users; managing, downloading, and displaying impact data stored in the device's memory; and transferring data from sensor devices to computer or cloud-based data storage, analytics, and reporting components.

In another embodiment of the present invention shown in FIG. 23, a single-unit charger containing a rechargeable battery is provided to store and charge a single device. The single-unit charger also contains a multi-channel radio transceiver to provide wireless connectivity and communications with the IMD device. In one embodiment of the present invention this wireless communications capability is implemented using the same multi-channel radio transceiver integrated circuit and dual band surface mount chip antenna as in the impact sensor device, capable of functioning in the 858 to 928 MHz ISM bands. In another embodiment of the present invention, a larger format, higher gain dual band 858 to 928 MHz antenna is integrated into the single-unit charger to provide longer range communications. The single-unit charger is further equipped with USB, Wi-Fi, or Bluetooth connectivity to communicate with software or browser applications running on smartphones, tablets, other mobile devices, PCs, remote hubs, routers, or servers, or in the cloud, as described above.

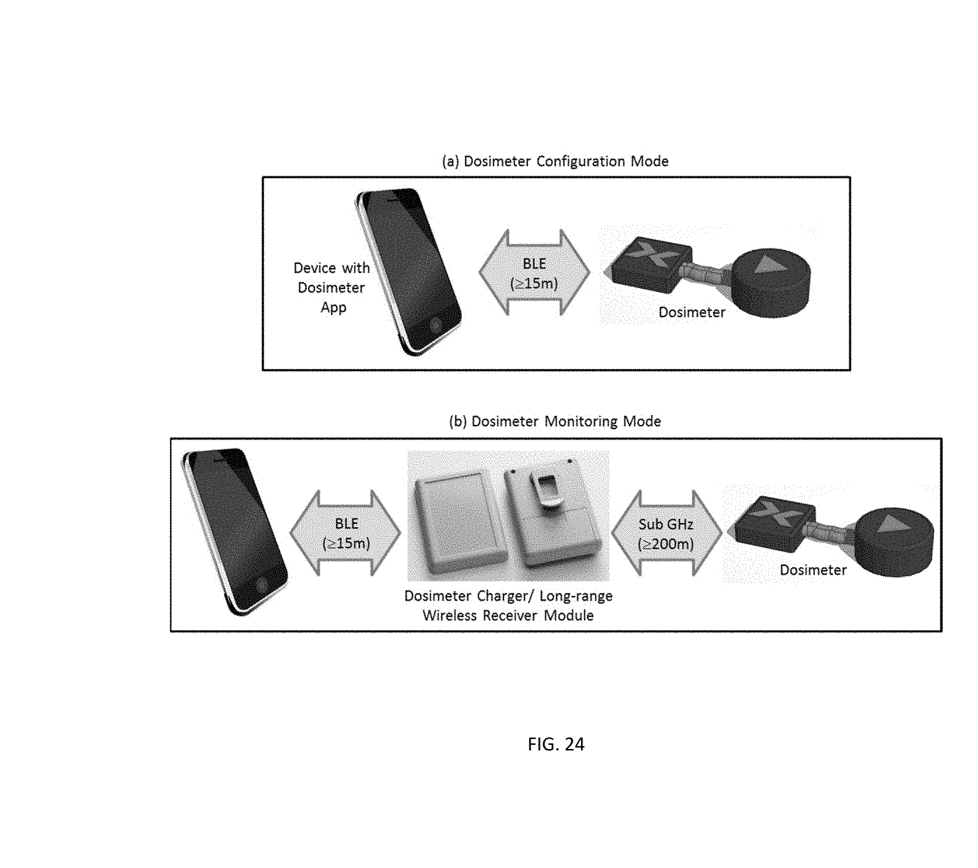

In another embodiment of the present invention shown in FIG. 24, the dosimeter charger/long-range wireless receiver may be configured as a clip-on module designed to be worn on the belt or clipped to a shirt pocket of supervisory personnel.

It is useful for the electronics system within the IMD to have a positional and axial frame of reference when attached to the wearer. In one embodiment of the invention, the enclosure may include a directional indicator to guide application of the IMD in a particular orientation, aiding in determining the axial reference frame of the IMD on the user. In other versions, the IMD may employ other visual, physical, or electronic means for determining an axial frame of reference.

A preferred IMD may optionally utilize outputs from the MEMS accelerometer and gyroscope, or from a separate orientation sensor, such as a MEMS magnetometer, together with algorithms stored in memory, to determine its orientation on the user. Low-cost MEMS orientation sensors are available that are small enough in size to be incorporated into a preferred embodiment of the IMD to provide information to the processor regarding the positional orientation of the device. The IMD would preferably also include a visual indicator of a preferred orientation for the device when attached to the wearer, for example indicating with an LED when the IMD is oriented to within ±5 degrees of each of the three perpendicular linear axes defining the orientation of the head when the user is standing vertically and facing straight forward toward magnetic north.

Additional sensors may also be incorporated into the IMD. One such sensor is an altimeter that can monitor the vertical position of the head with respect to the ground with centimeter accuracy, and quickly determine whether the user has experienced a fall in conjunction with an impact event.

Another such sensor is a magnetometer that can monitor the horizontal directional orientation of the head with respect to magnetic north with degree accuracy, to aid in establishing an absolute reference orientation for the device on the user's head.

Another such sensor is a thermometer configured to detect the temperature of the surface on which the IMD is mounted. Preferably, the thermometer is positioned sufficiently close to the user-facing surface of the IMD, or through an opening in the adhesive, such that the thermometer will detect the temperature of the wearer at the location of the IMD. In one version, the detected temperature may be used to determine IMD proximity to the skin and therefore whether the IMD is attached to a user. In other versions, the thermometer data is collected and associated with impact sensor data to facilitate evaluation of the overall health of the wearer, or the safety of the user's surrounding environment.

Further embodiments of the IMD may include a heart rate sensor. As with a temperature sensor, the heart rate sensor may be used to detect the presence of a pulse of the wearer and thereby confirm that the IMD is positioned on a living person. In addition, heart rate data may be collected by the IMD and stored in the memory to track the user's heart rate, particularly at times before and after an impact event.

An additional embodiment of the IMD may include a hydration sensor such as a low-cost, small microelectromechanical MEMS sensor. The hydration sensor is positioned in the IMD to make sufficient contact with the skin in order to detect the hydration of the wearer, preferably by being configured to extend through an opening in the adhesive. Similarly, the IMD may include one or more chemical sensors to detect and enable evaluation of the concentration of electrolytes, metabolites, brain injury biomarkers, or other chemical substances that may be present in the user's perspiration or subdural blood flow. Impact injury thresholds may be modified in real time during the user's activities as a function of the chemical levels measured with the above sensors.

In another embodiment of the present invention, remove-from activity and safe return-to-activity status are determined by utilizing one or more components of the inherent body sway power or statistical gait irregularities, alone or in combination, as a physio-mechanical biomarkers whose values are each compared to a baseline value for the user stored in the device for both single impact events and cumulative sequences of impacts. The baseline values may be determined directly from postural sway and gait measurements of the user made with the IMD, or may be based on postural sway and gait data for a generalized population of similar users. Deviations from the baseline values are used to assess the degree of accumulation of permanent physiological changes and the degree of recovery from transient physiological changes, to generate alerts when a user should be removed from activity, and to generate alerts when a user can return to active following sufficient recovery from transient physiological changes.

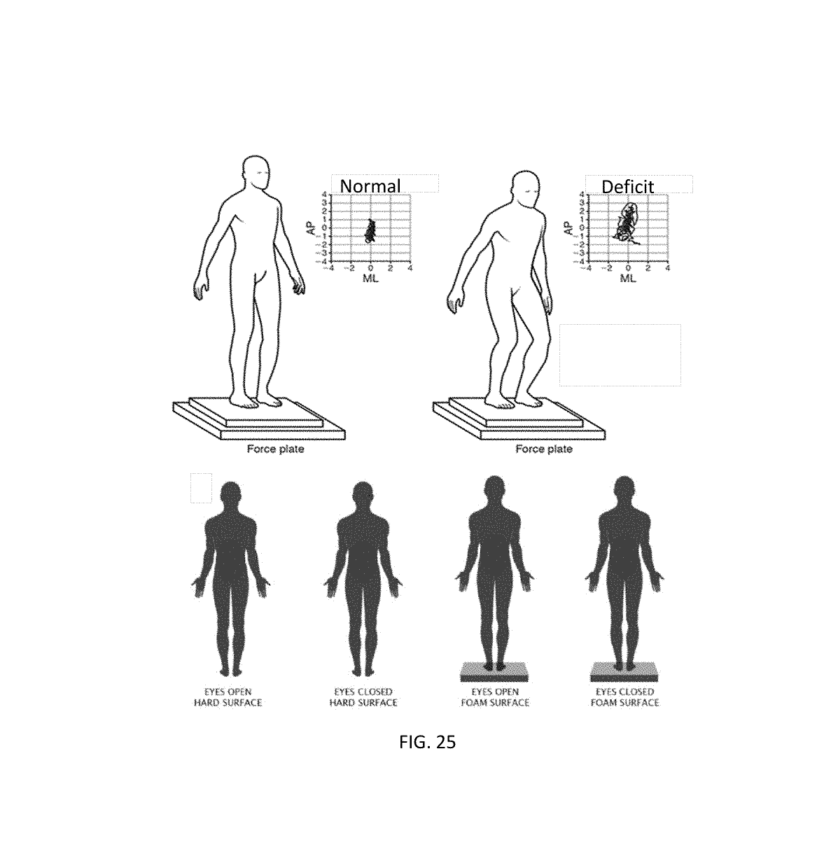

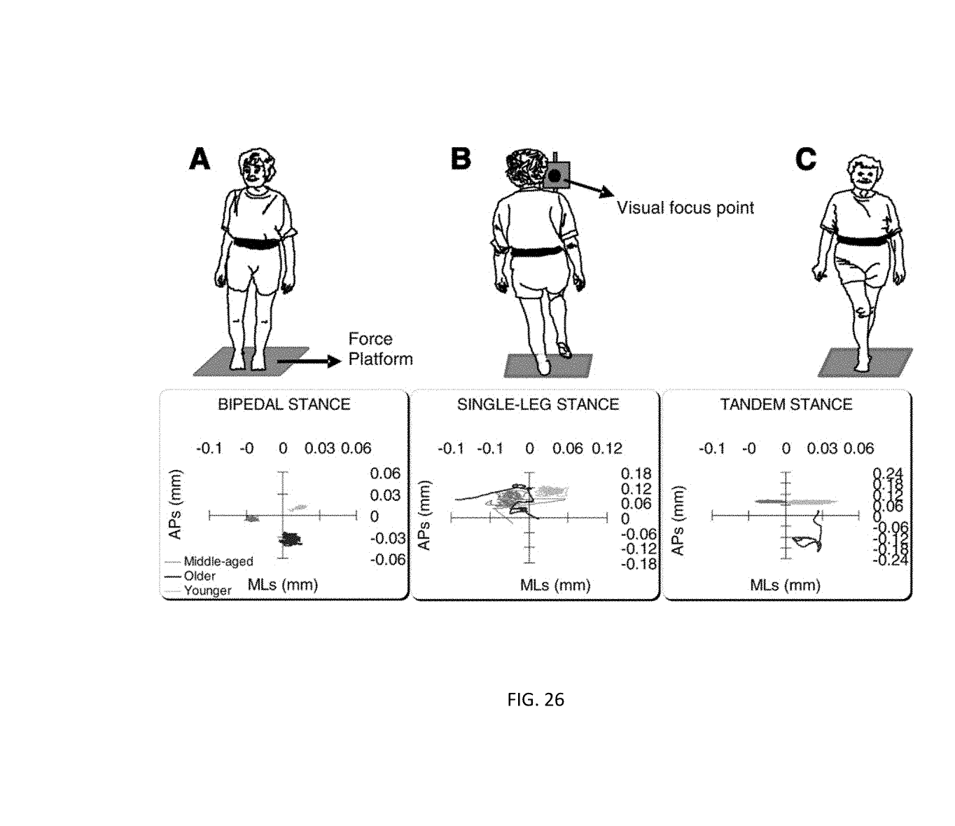

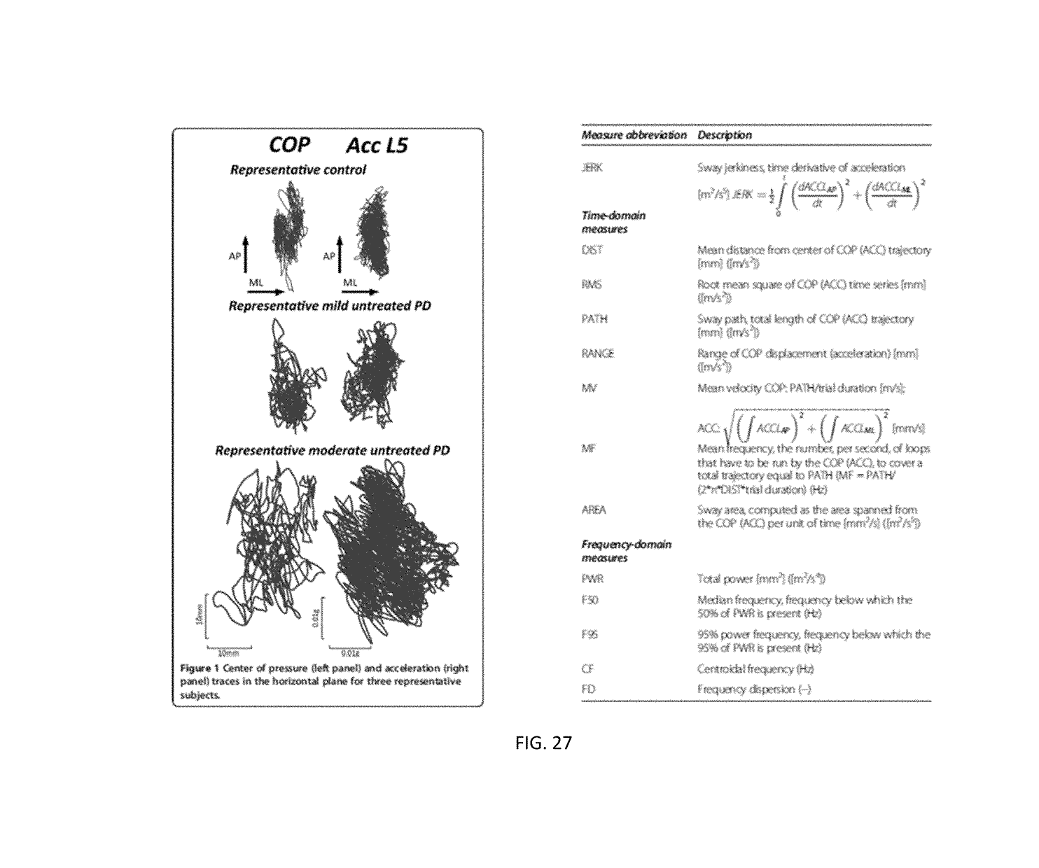

Balance impairments due to vestibular dysfunction are triggered by repetitive sub-concussive head impacts at levels that can be detected via postural sway measurements using a variety of instruments. As illustrated in FIG. 25, such measurements were initially developed using force plate analysis of center of center of pressure (COP) displacements using a variety of stances, often with eyes opened and closed, and on hard surfaces and soft foam surfaces. Studies of postural control have indicated that characterization of medial-lateral (ML) and anterior-posterior (AP) body sway, as illustrated in FIG. 26, in amplitude, smoothness, and frequency is applicable to quantifying balance deficits arising from a wide range of neurological and musculoskeletal conditions, including Parkinson's disease, Huntington's disease, multiple sclerosis, amyotrophic lateral sclerosis (ALS), chronic traumatic encephalopathy (CTE), stroke, and concussions. More recently, body worn accelerometers have been shown to be a more portable alternative to force plates for measurement of postural sway. These measurements are typically made using triaxial accelerometers worn in a belt at the level of the sacrum, which is close to the body's center of mass. FIG. 27 illustrates sample postural sway data from a force plate and a body-worn accelerometer, and lists a wide range of metrics extracted from postural sway data to quantify various motion disorders. Total sway power and sway “jerkiness”, the derivative of the linear acceleration, are found to show the best test-retest reliability and correlation with parallel clinical assessments. Camera-based postural sway analyses have indicated that measurable transient balance control deficits are caused by 10 sub-concussive impacts (heading a soccer ball at a linear acceleration of 14.5 g) over a period of 10 min, and that these changes returned to baseline levels after 24 hours.

More recently, studies of postural sway in healthy individuals and patients suffering from Parkinsons's disease have demonstrated very good correlation between motion data measured using head-mounted and belt-mounted accelerometers, indicating that an IMD can be utilized to directly measure and correlate motion disorders resulting from cumulative head impact loads measured by the same device.

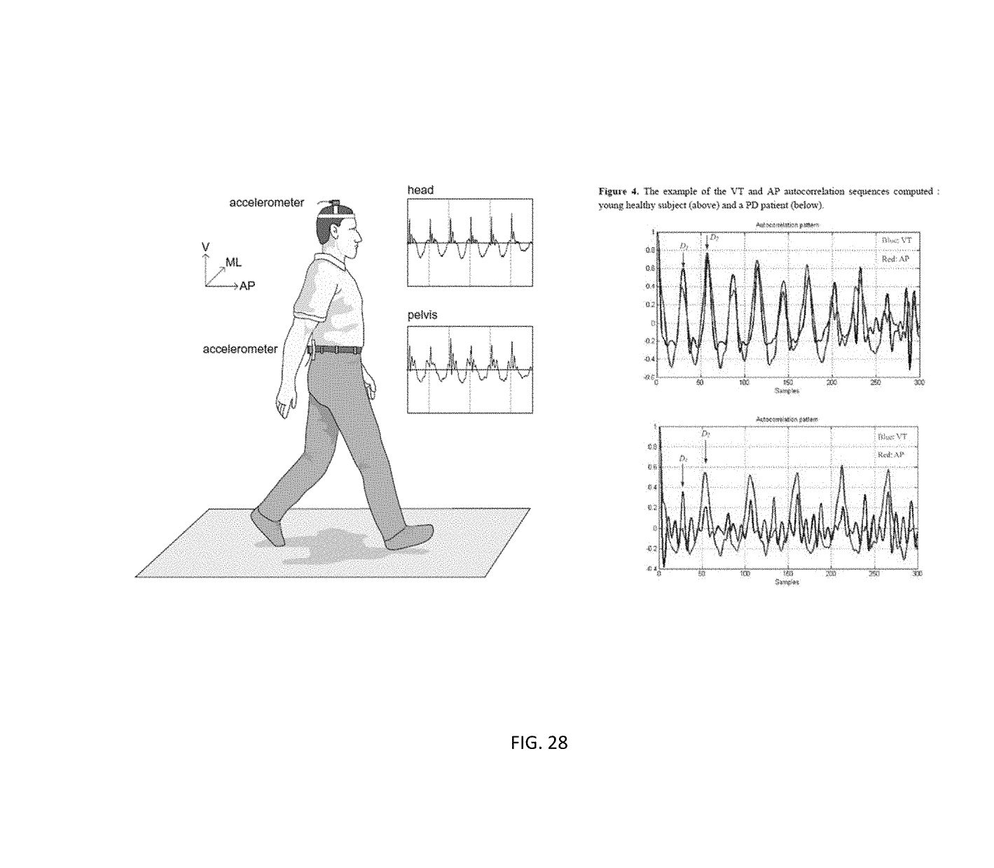

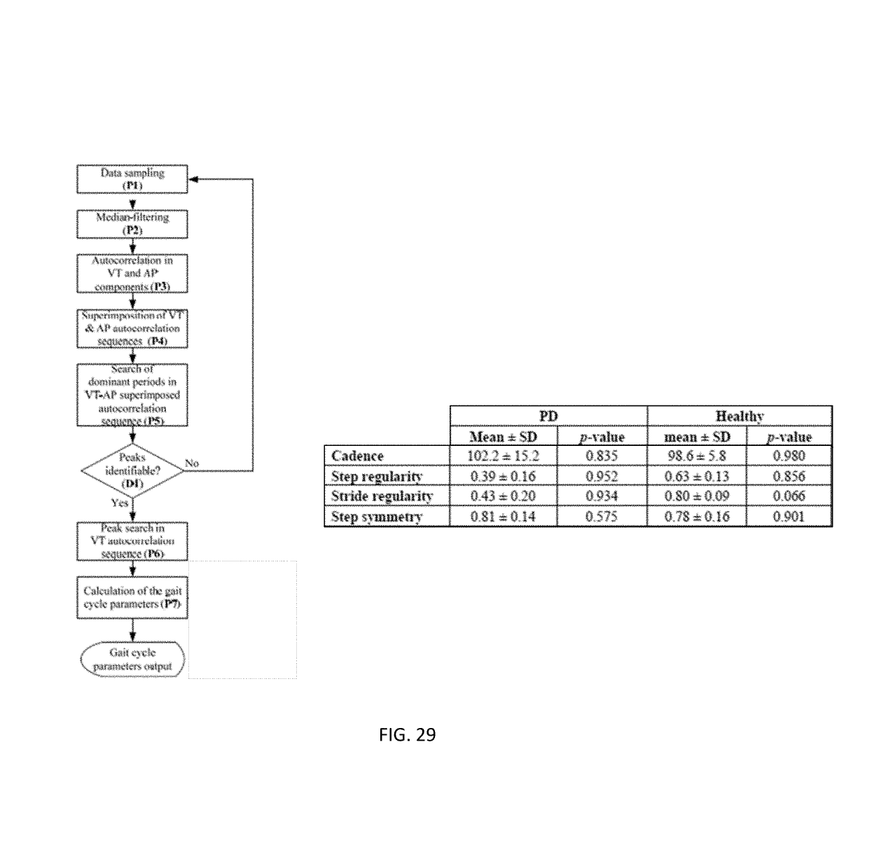

Wearable sensor data has also shown that variations in the step-to-step and stride-to-stride regularity of medial-lateral (ML), anterior-posterior (AP), and vertical (V) head accelerations provide a sensitive complementary measure of motion disorders when the user is in motion, as illustrated in FIG. 28. These variations can be directly measured by the IMD using the same algorithms as presented in FIG. 19, along with the process flow illustrated in FIG. 29.

The present invention enables significant advances and improvements in real-time monitoring of impact injury risks by allowing supervisory personnel to continuously monitor balance control and motion deficits and correlate these changes in real time with cumulative head impact power, using a single IMD device that can calculate:
1. head impact power when an impact is detected by the IMD;
2. postural sway power when a period of quiet stance is detected by the IMD; and
3. variations in step-to-step and stride-to-stride acceleration when the user is determined by the IMB to be walking or running.

In one embodiment of the invention, when the user is determined by the IMD to be in a quiet stance, an audio alert can be given to the user that a postural sway measurement is about to begin and they should remain standing in place for a predetermined period of time, for example between 10 seconds and 60 seconds. A second audio alert can be given to the user when the measurement period has ended, so that they no longer need to remain standing in place.

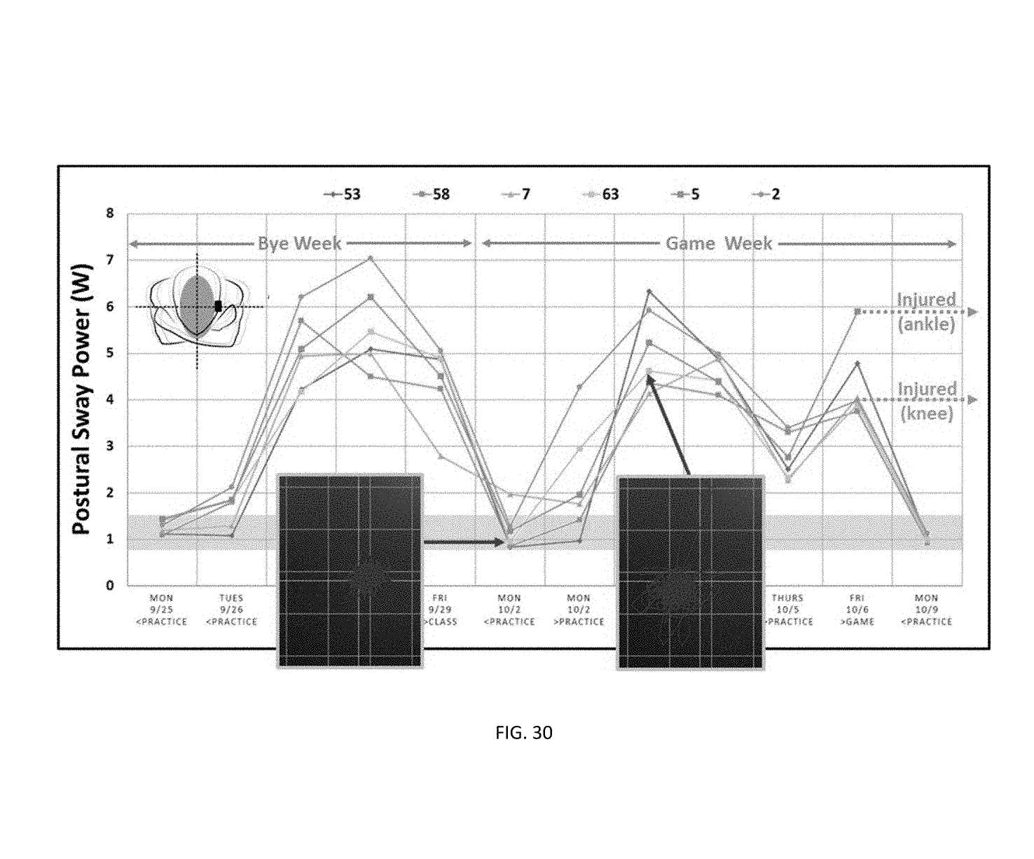

FIG. 30 shows sample postural sway data from an IMB, along with calculated total sway power vs. time for sub-concussive head impact exposure, recorded for six high school football players over a two-week period. In addition to allowing supervisory personnel to monitor and limit cumulative head impact exposure in order to ensure regular return of postural sway performance to baseline levels, the current invention also provides guidance indicative of increased risks of injuries to other body parts, such as ankles or knees, which manifest themselves as asymmetries or off-center displacements in the postural sway data.

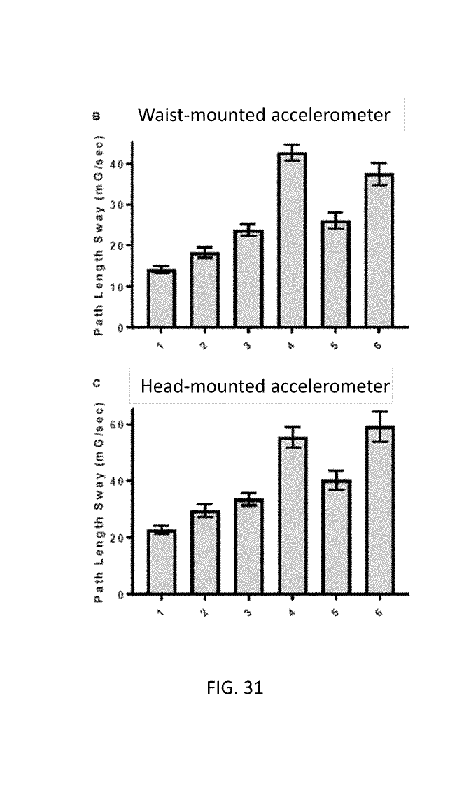

One benefit of the current invention over waist/torso-mounted sensors and force plates is enhanced sensitivity to abnormal strategies of head stabilization following repetitive head impacts, which lead to en bloc movement of the head and upper torso and faster angular velocities/higher total sway powers for the head and upper torso, illustrated by the larger total sway path length observed for head-mounted vs. waist-mounted accelerometers in FIG. 31. An additional benefit is that postural sway measurements repeated at multiple times throughout training or competition activities provide sufficient statistical accuracy from a single bipedal stance with eyes open on standard playing surfaces.

A further benefit of the present invention is that direct measurement by an IMB, throughout training or competition activities, of both total sway power when the user is determined by the IMD to be in a quiet stance, and variations in the regularity of step-to-step and stride-to-stride acceleration when the user is determined by the IMB to be walking or running, allows more accurate isolation of motion deficits due to cumulative head impacts versus those due to other confounding variables such as physical exhaustion and dehydration, together with a more comprehensive multi-variate determination of objective remove-from-activity and return-to-activity conditions.

In another embodiment of the present invention, remove-from activity and safe return-to-activity status are determined by utilizing one or more components of eye-tracking motion, including fixations, saccades, vergence movements, smooth pursuit, and the vestibular-ocular reflex, alone or in combination, as neurophysiological biomarkers whose values are each compared to a baseline value for the user stored in the device for both single impact events and cumulative sequences of impacts. The baseline values may be determined directly from corrected eye-tracking measurements of the user made with an IMB and eye-tracking device (ETD), or may be based on eye-tracking data for a generalized population of similar users. Deviations from the baseline values are used to assess the degree of accumulation of permanent physiological changes and the degree of recovery from transient physiological changes, to generate alerts when a user should be removed from activity, and to generate alerts when a user can return to activity following sufficient recovery from transient physiological changes.

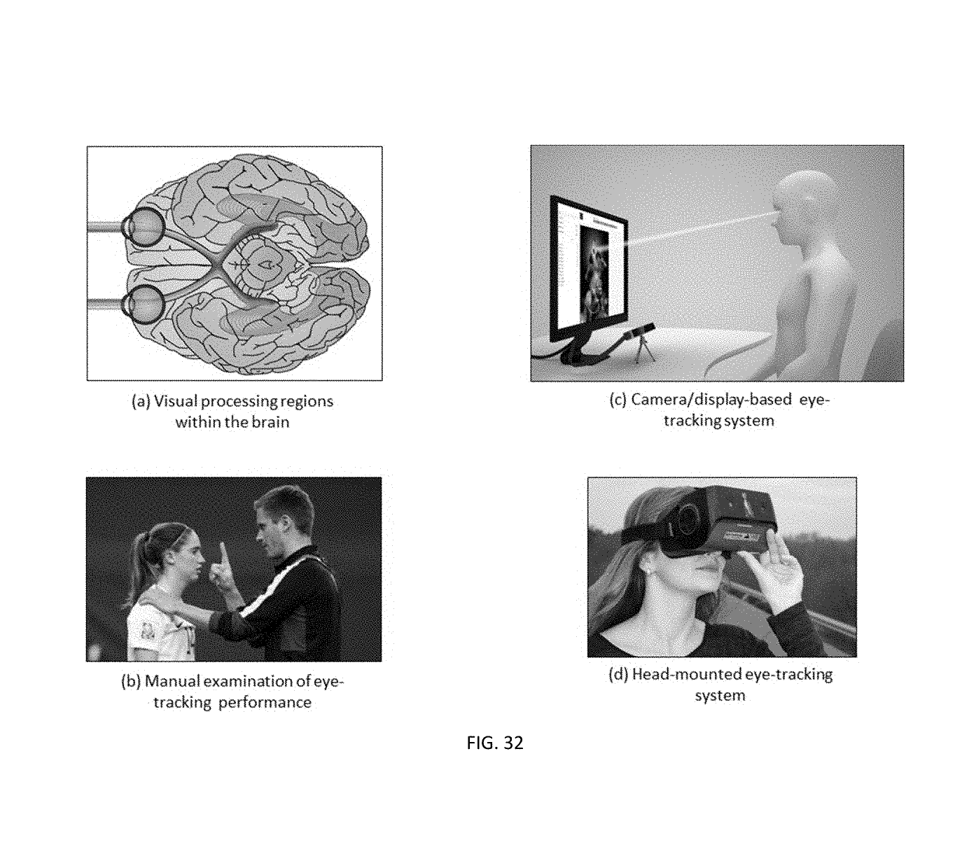

As illustrated in FIG. 32(a), visual processing is widely distributed throughout the brain. As a result, localized damage to neural tissues in many regions of the brain will impact visual performance. Resulting changes in eye-tracking components such as vergence movements and smooth pursuit can be observed via manual examination, as illustrated in FIG. 32(b). More precise measurements and analyses of eye tacking motion components, including fixations, saccades, vergence movements, smooth pursuit, and the vestibular-ocular reflex, require systems that combine display screens and eye-tracking cameras, or other eye-tracking sensors, as illustrated in FIG. 32(c), or integrate these components into head-mounted eye-tracking systems, as illustrated in FIG. 32(d).

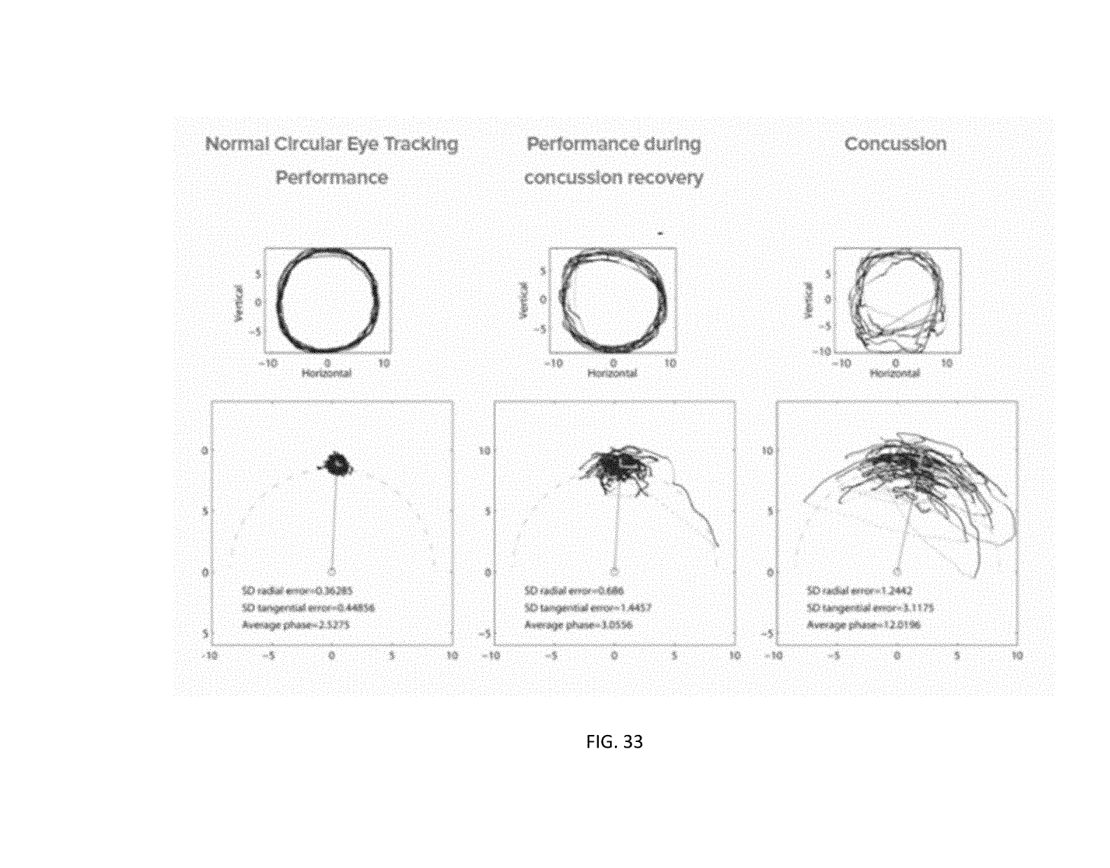

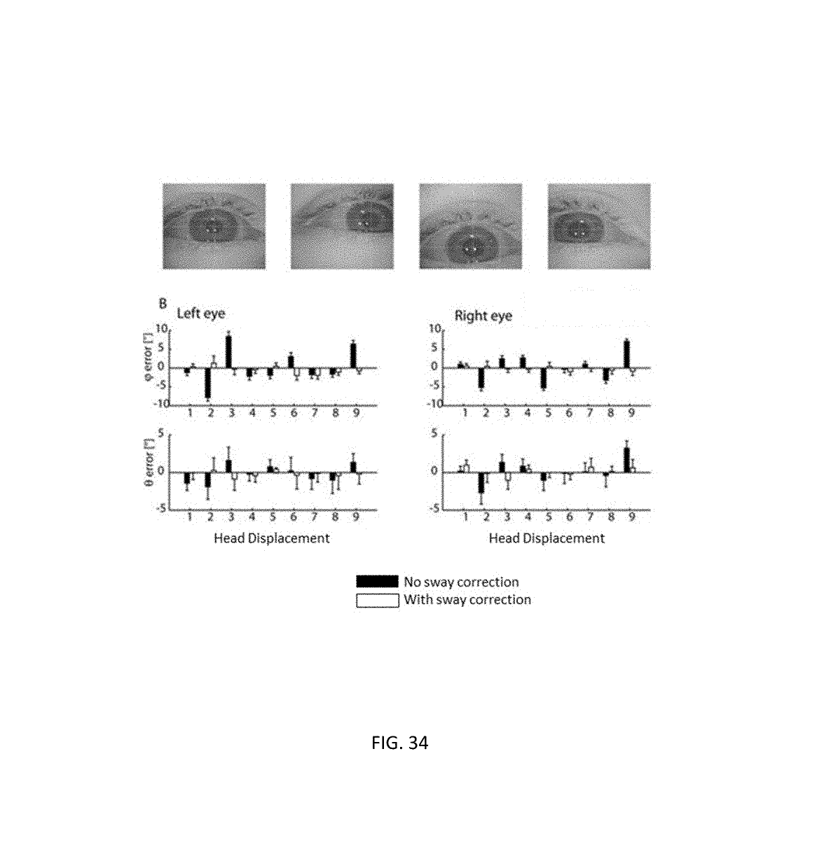

FIG. 33 shows sample eye-tracking data for normal performance, performance immediately following a concussion, and performance at a later point in time during concussion recovery. For the case illustrated in FIG. 33, the user's eyes are tracking a visual target that is moving along a circular path, and the eye-tracking system is measuring motion errors parallel to the instantaneous path of the target (radial errors), motion errors perpendicular to the instantaneous path of the target (tangential errors), and the delay between the motion of the eyes and the motion of the target (phase errors). However, as has already been illustrated in FIG. 30, significant postural-sway-induced motion of the head also results from concussions and sub-concussive injuries. Since the vestibular-ocular reflex causes the eyes to move involuntarily in the opposite direction of the head, motion of the head due to balance impairments and other vestibular-motor deficits is a confounding variable when utilizing eye-tracking measurements to quantify and monitor head impact injuries. In the present invention, direct measurements of the motion of the head provided by the IMD are utilized to calculate corrected eye-tracking movements. Since the speed of the eye normally equals the speed of the head, but in the opposite direction, one correction that can be applied to measurement data such as that illustrated in FIG. 33 is to subtract out the motion of the head, which typically reduces the measured radial, tangential, and phase errors, yielding corrected values indicative of localized neural tissue damage within the brain separate from impact induced impairments to the vestibular-motor and vestibular-ocular systems. FIG. 34 illustrates results of such a correction process.

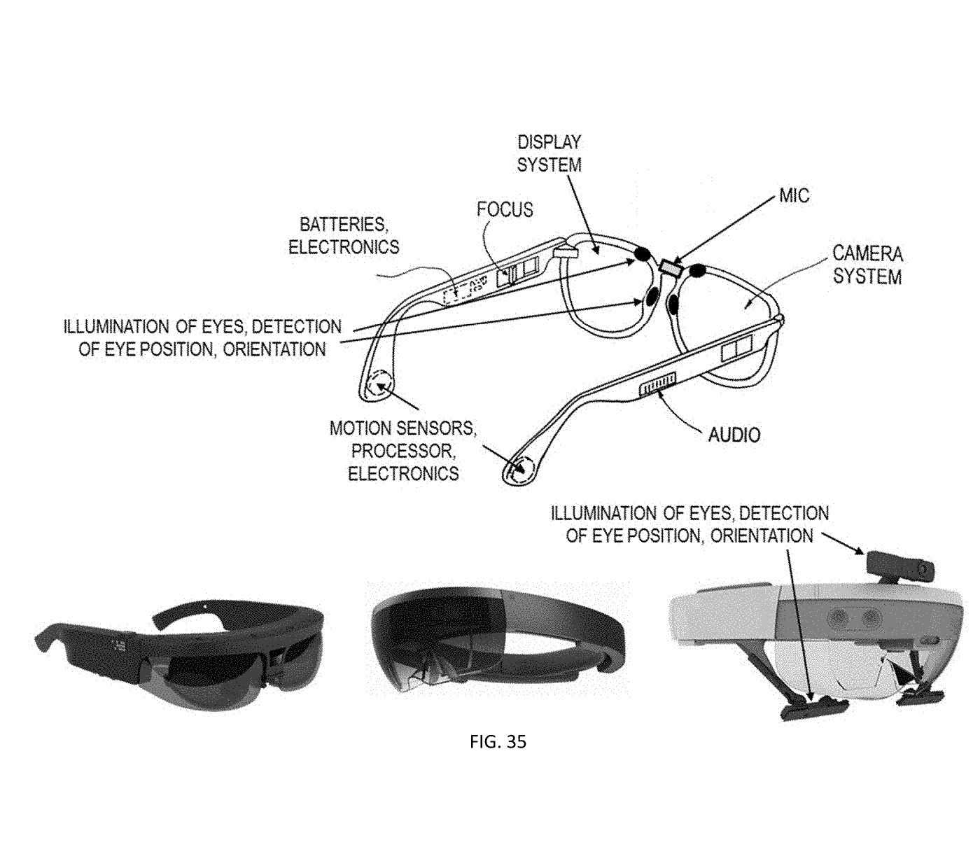

FIG. 35 illustrates another embodiment of the current invention, in which sensors for cumulative head impact monitoring, postural sway measurements, and corrected eye-tracking measurements are integrated into a head-mounted system. Such a head mounted system includes one or more of the following components integrated into a frame or a goggles device: motion sensors, a display system, a display focus control, a camera system to monitor the position and orientation of the eyes, infrared or visible emitters and detectors to illuminate and detect the position and orientation of the eyes, one or more audio speakers, audio microphone, processor electronics, electronic storage medium, and one or more batteries.

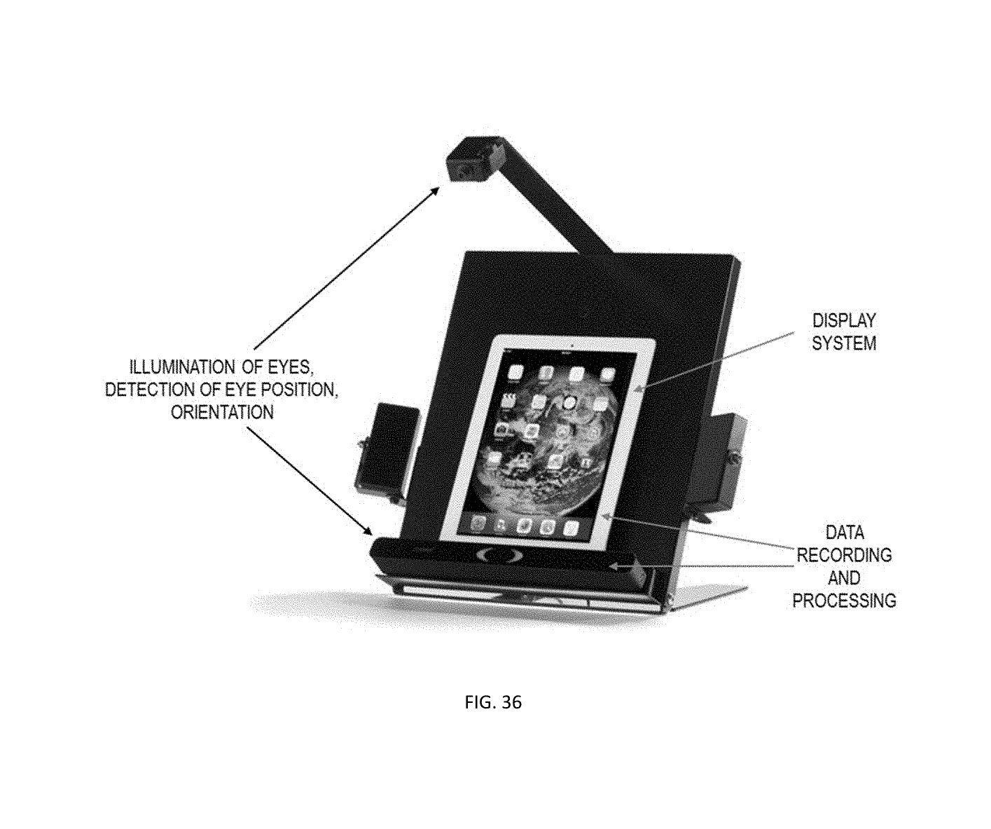

FIG. 36 illustrates another embodiment of the current invention, in which components other than the head-mounted motion sensors are combined together with a smartphone, tablet computer, or laptop computer into a mobile system for cumulative head impact monitoring, postural sway measurements, and corrected eye-tracking measurements suitable for deployment on the sidelines of live athletic events.

In another embodiment of the present invention, remove-from activity and safe return-to-activity status are determined by utilizing one or more components of corrected eye-tracking motion, including fixations, saccades, vergence movements, smooth pursuit, and the vestibular-ocular reflex, alone or in combination, to derive a neurophysiological biomarker that can quantify deficits in the user's ability to direct their visual attention. The measured value of this neurophysiological biomarker is compared to a baseline value for the user stored in the device for both single impact events and cumulative sequences of impacts. The baseline values may be determined directly from corrected eye-tracking measurements of the user made with an IMD and eye-tracking device (ETD), or may be based on eye-tracking data for a generalized population of similar users. Deviations from the baseline values are used to assess the degree of accumulation of permanent physiological changes and the degree of recovery from transient physiological changes, to generate alerts when a user should be removed from activity, and to generate alerts when a user can return to activity following sufficient recovery from transient physiological changes.

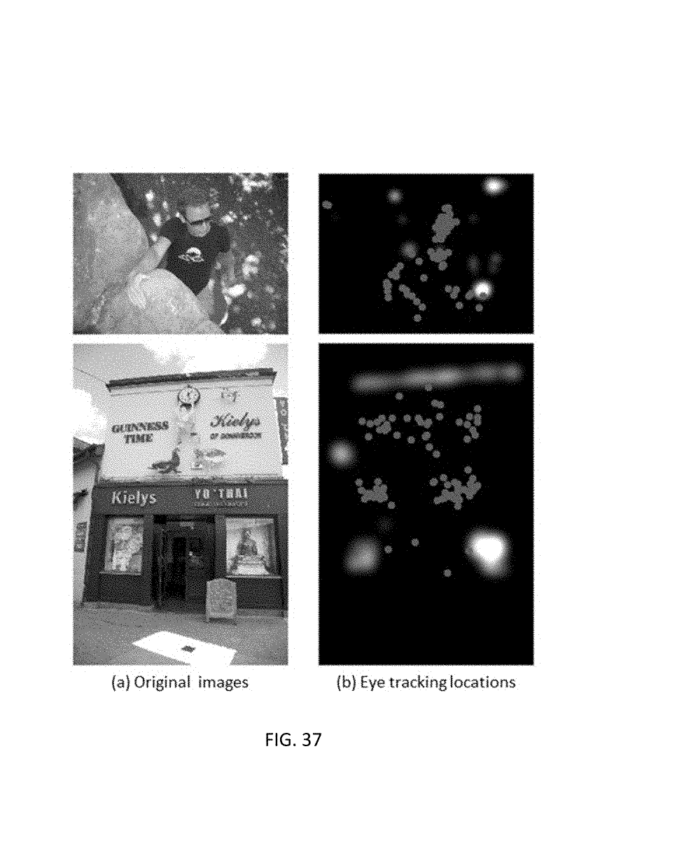

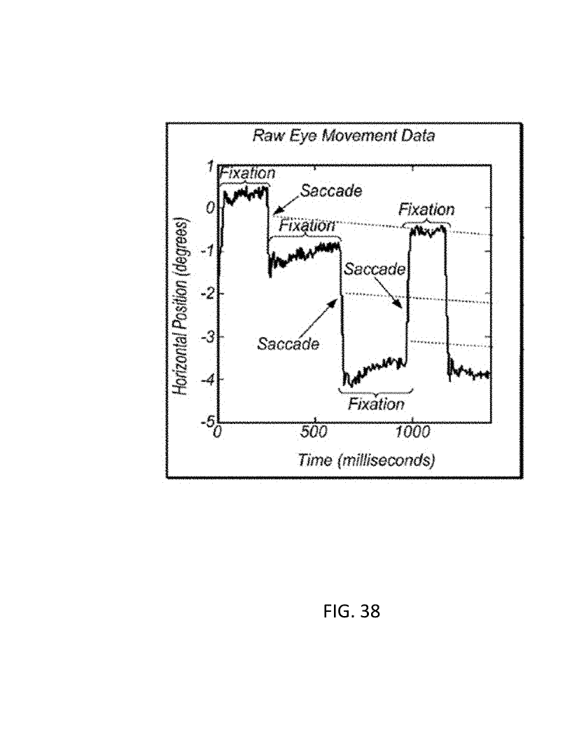

As illustrated in FIG. 37, the locus of human visual attention within a scene consists of a series of fixation locations. In natural scene viewing, where the visual environment is more complex than in the simple eye-tracking experiments shown in FIG. 33, eye tracking provides a very accurate guide to the locus of attention. Fixation locations are determined by low-level visual features such as intensity, color, orientation, texture, and motion, along with the presence of familiar objects such as faces, people, body parts, animals, text, and cars, which combine multiple low-level visual features. Since visual scenes typically contain many objects that compete for the control of attention and eye movements, individual fixation episodes are linked by gaze tracking paths consisting of a series of saccades, as illustrated in FIG. 38.

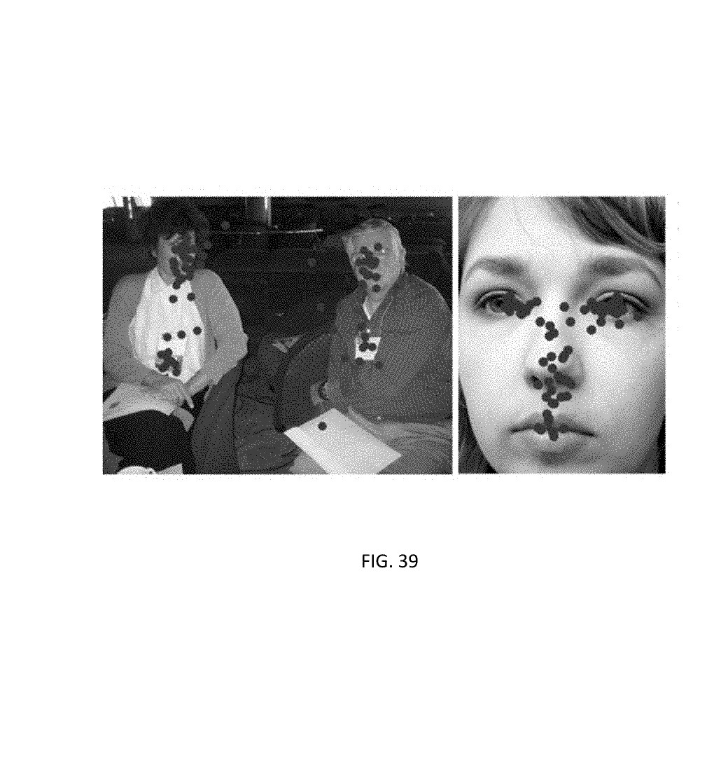

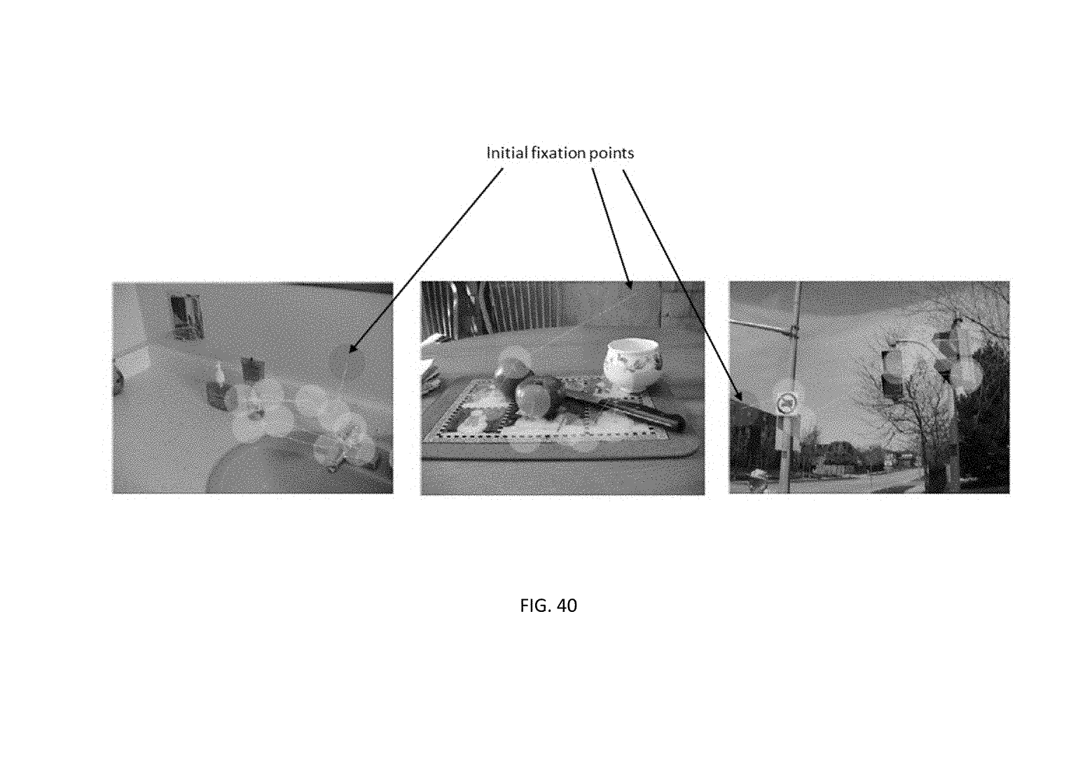

Large-scale quantitative analyses of fixation points and gaze paths provide ground truth data for research into visual perception, and large collections of images and eye-tracking data are available in databases that have been made available for vision and graphics research communities. This ground truth data reveals many visual attention strategies common to all viewers. For example, as illustrated in FIG. 39, viewers fixate on human faces when they are present in images. When a face fills a large portion of the image, viewers utilize a “T-shaped” visual attention strategy to focus on the eyes, nose, and lips. Even in more complex images, as illustrated in FIG. 40, following an initially random gaze point, users all tend to focus on similar collections of features that the human visual processing system has evolved to consider as biologically relevant.

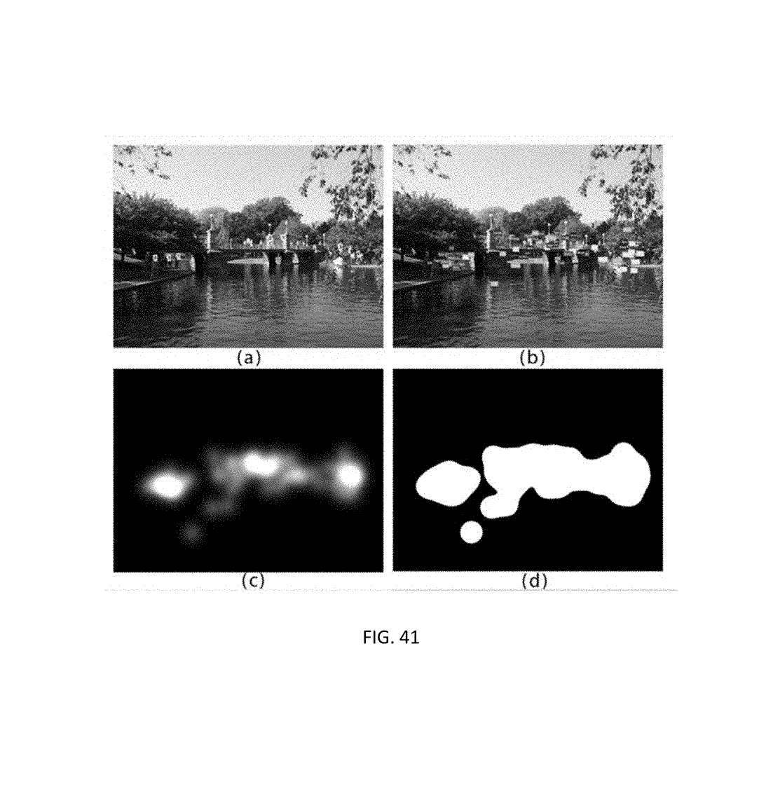

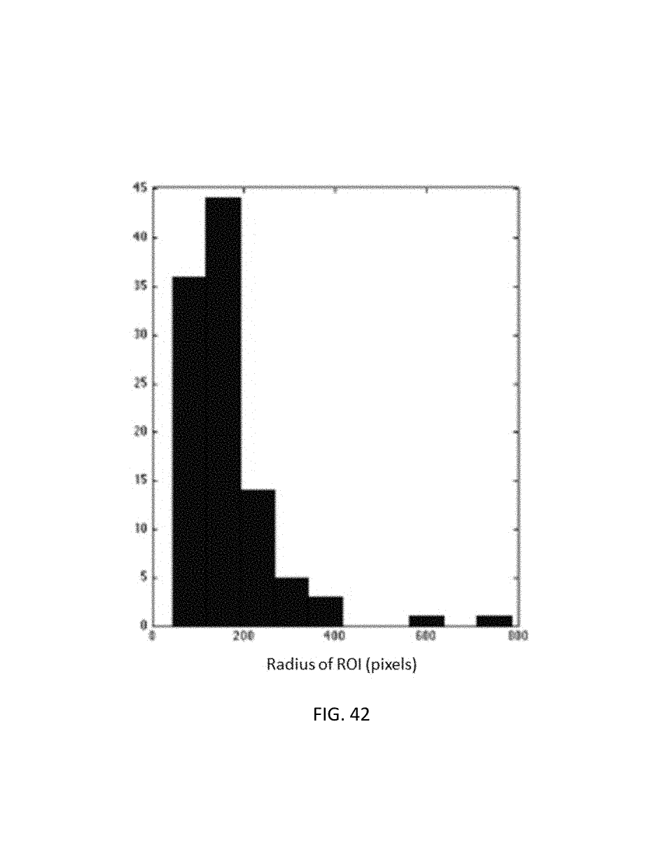

In one embodiment of the current invention, eye-tracking is utilized to detect deficits in the user's visual attention that lead to changes in coverage area of the ground truth scan path within static and moving images presented for viewing. The basic steps involved in this process are illustrated in FIG. 41. We collect eye-tracking data from users using images selected from a large database for which ground truth eye-tracking data is also available (FIG. 41a). Baseline gaze tracking paths and fixation locations are recorded for each user (FIG. 41b) prior to each event during which head impact exposure may occur. A continuous “visual relevance map” (FIG. 41c) is found by calculating the convolution of a spatial Gaussian filter over the ground truth fixation locations and the user's baseline fixation locations. The Gaussian mean is centered on the geometrical mean calculated for each fixation location, while the Gaussian standard deviation, in pixels, is selected based upon measured histograms of the radii of the regions of interest (ROI) centered about each fixation location (FIG. 42). The Gaussian standard deviation can be varied for each fixation location, based on ground truth histograms of the radii, as well as from image to image, based upon each image's pixel dimensions. The visual relevance map is then thresholded to produce a binary map that encompasses a fractional area of the image, such as 10 percent, containing the most visually relevant regions of the image (FIG. 41d). One or more initial fixation points in each scan path, shown in FIG. 40, are discarded from each scan to eliminate this potentially random information from the initial point or points of fixation.

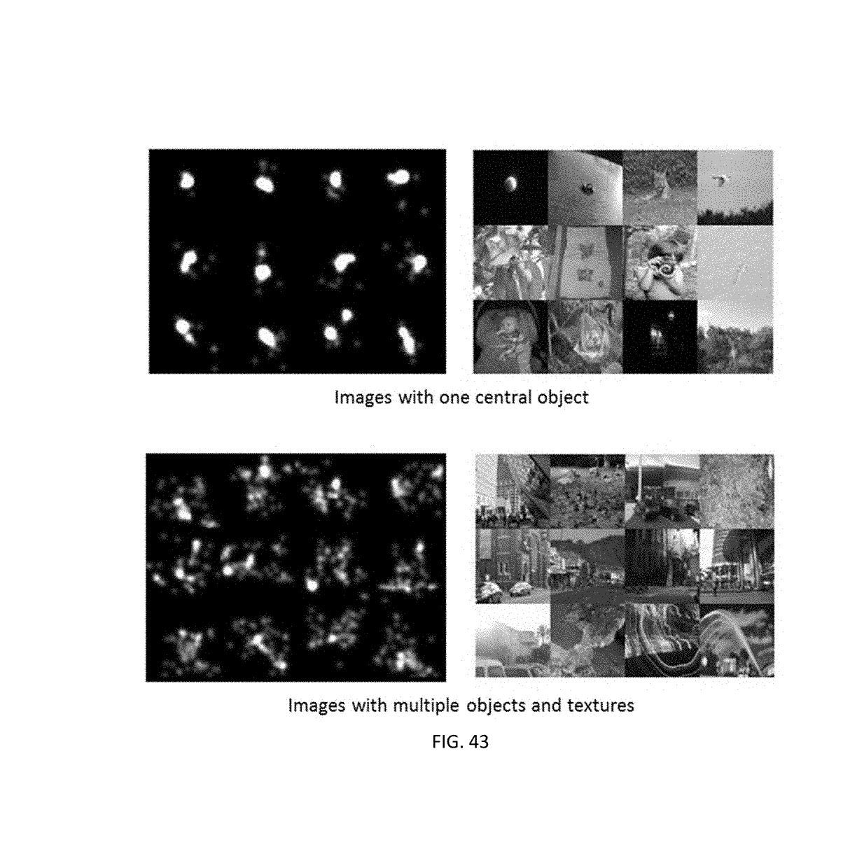

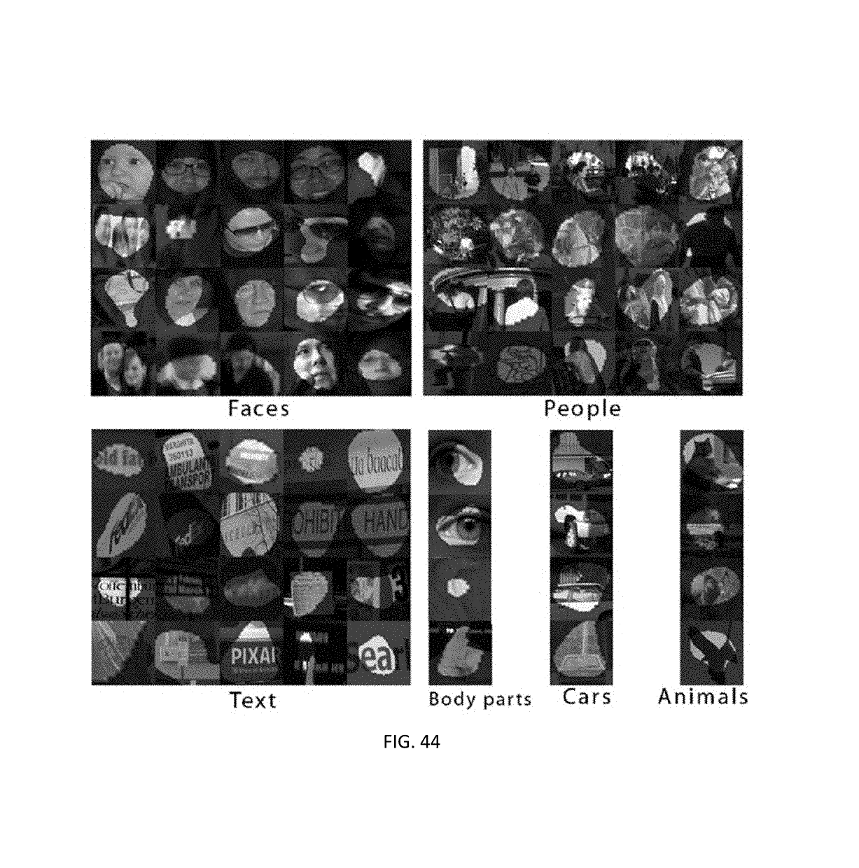

In order to overcome the strong bias for human fixations to be near the center of the image, and to present opportunities for users to fixate on the same location within an image as well as to disperse viewers' fixations more widely over the image, we utilize a combination of images with one central object, as well as images with multiple objects and textures (FIG. 43). In order to present images that can effectively test visual deficits across large user populations, we utilize images containing universally recognized objects such as faces, people, body parts, animals, text, and cars, which combine multiple low-level visual features (FIG. 44).

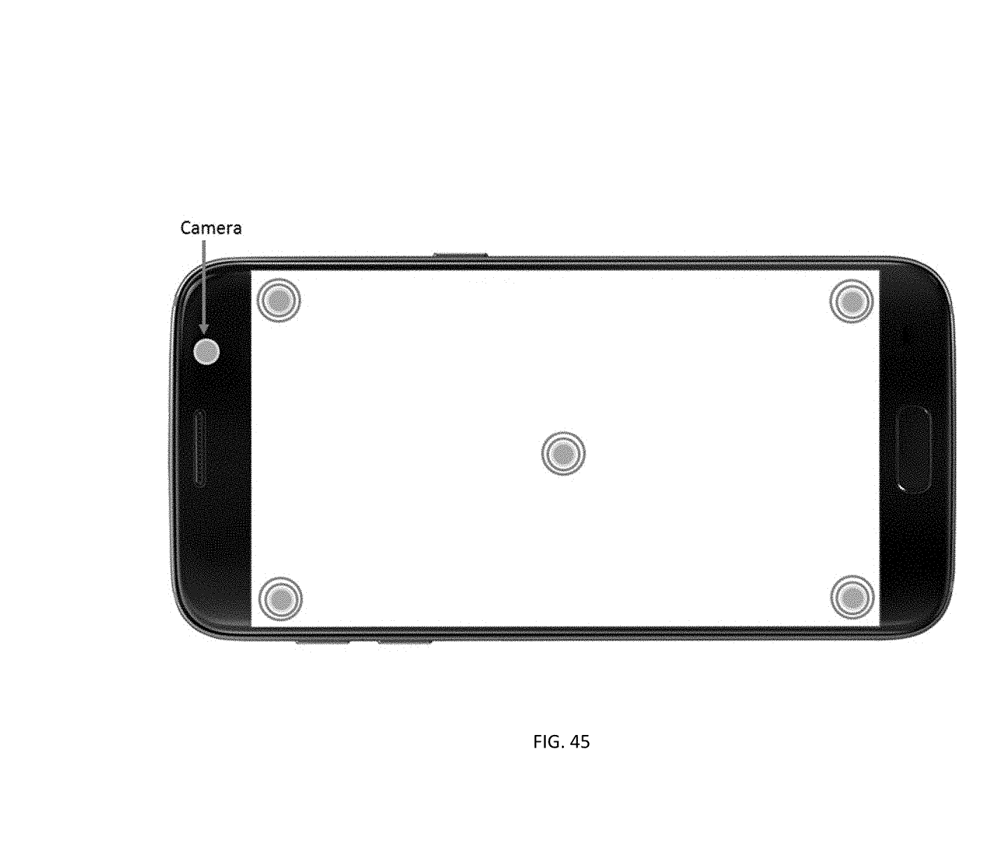

At the beginning of each eye-tracking test, a calibration procedure is carried out in which the user's attention is directed to 5 points on the screen (FIG. 45). The eye-tracker then reports where the user is looking relative to the camera or detector for each of these 5 points, and the resulting map of 5 coordinate pairs is subsequently used to calculate the gaze position when images are presented on the screen.

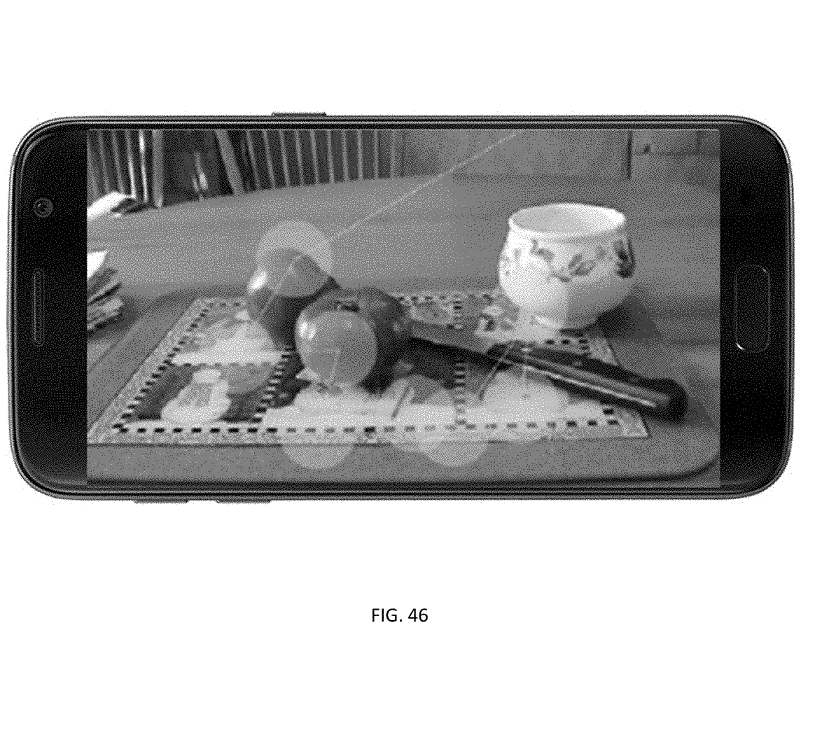

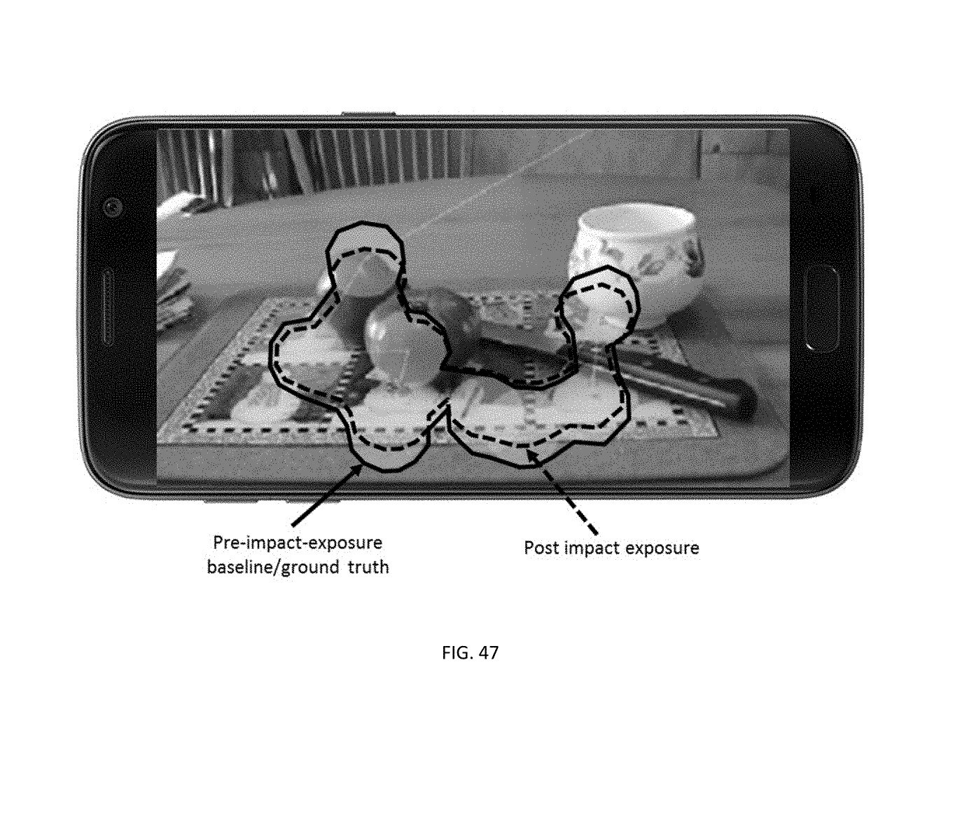

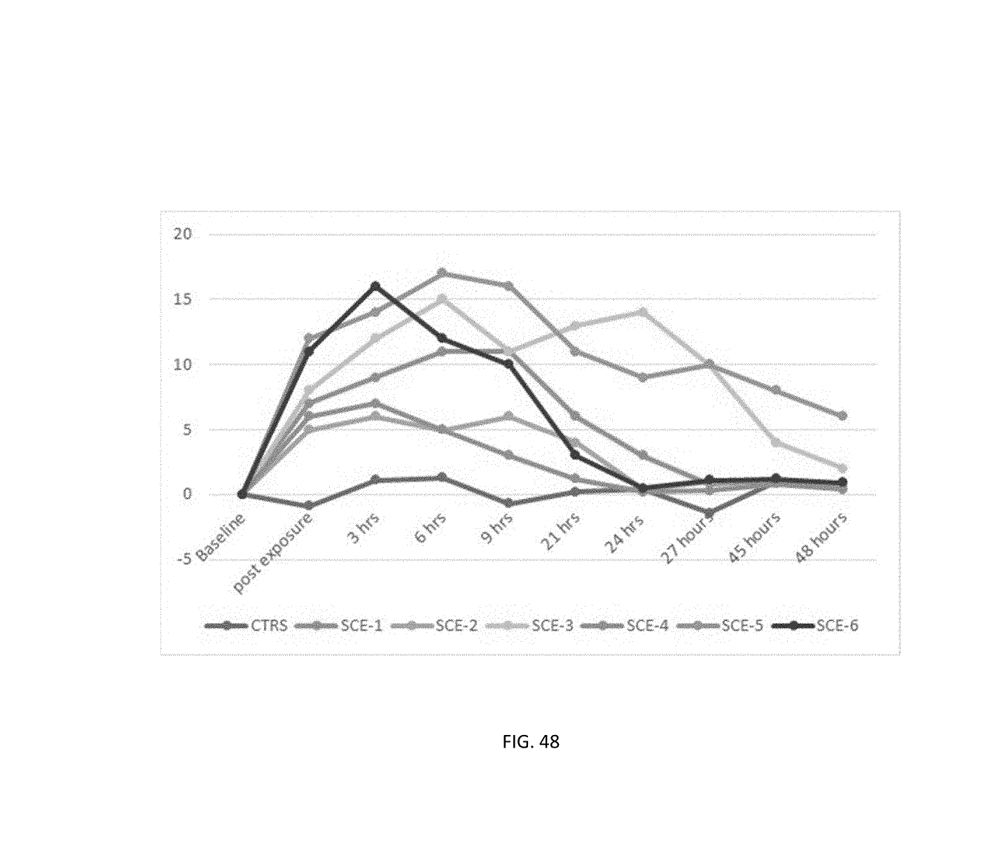

In one embodiment of the present invention, following head impact exposure, the user is presented with a series of 10 static images, as illustrated in FIG. 46. Each image is viewed for 1-3 seconds, separated by 0.5-1 second viewing a grey screen. A binary visual relevance map is then calculated for each of the 10 images post impact exposure, and compared to the pre-impact exposure binary visual relevance map or ground truth map for the same image, as illustrated in FIG. 47. The areas of the pre-impact exposure and post-impact exposure maps, and the percentage difference between the areas, are then calculated for each of the 10 images. The average of these percentage area differences for all 10 images is then calculated and plotted as a function of time following head impact exposure. FIG. 48 illustrates these average percentage area differences for six users exposed to sub-concussive head impact loads, compared to the average percentage changes observed for six control subjects. Analogous to the postural sway results illustrated in FIG. 30, the eye tracking results illustrated in FIG. 48 can be utilized to provide both remove-from activity and safe return-to-activity guidance.

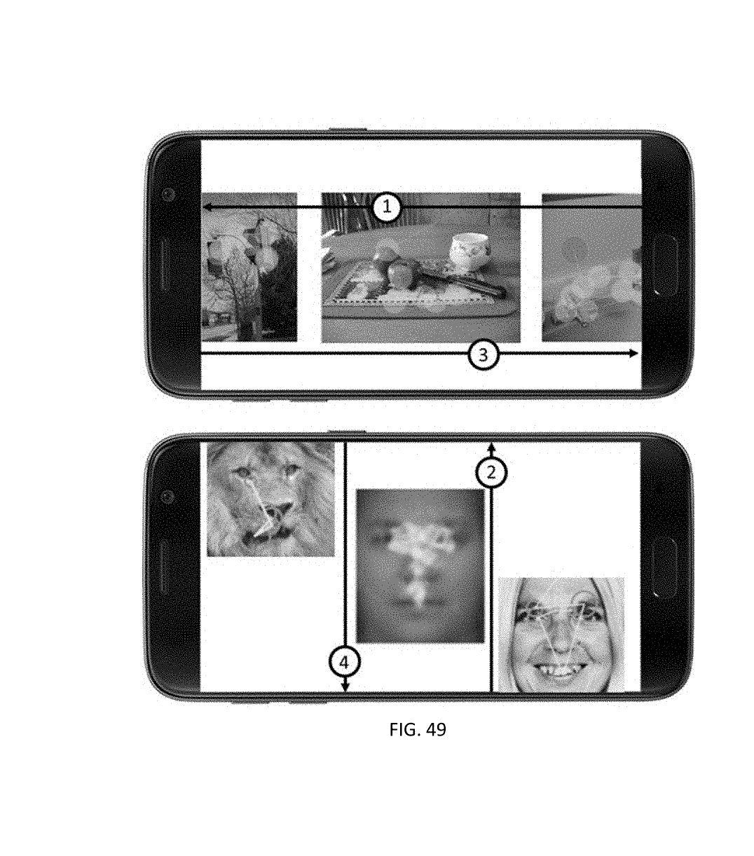

In another embodiment of the present invention, following head impact exposure, the user is presented with four series of three images each moving across the screen, as illustrated in FIG. 49. In the first series, the images move across the screen from right to left, and each image transits the screen over a time period of 0.5-3 seconds. When the second image has reached the center of the screen, the last half of the first image and the first half of the third image are also present. In the second series, the images move across the screen from bottom to top, and each image transits the screen over a time period of 0.5-3 seconds. When the second image has reached the center of the screen, the last half of the first image and the first half of the third image are also present. In the third series, the images move across the screen from left to right, and each image transits the screen over a time period of 0.5-3 seconds. When the second image has reached the center of the screen, the last half of the first image and the first half of the third image are also present. In the fourth series, the images move across the screen from top to bottom, and each image transits the screen over a time period of 0.5-3 seconds. When the second image has reached the center of the screen, the last half of the first image and the first half of the third image are also present. A binary visual relevance map is then calculated for each of the 12 images post impact exposure, and compared to the pre-impact exposure binary visual relevance map or ground truth map for the same image. The areas of the pre-exposure and post-exposure maps, and the percentage difference between the areas, are then calculated for each image. The average of these percentage area differences for all 12 images is then calculated and plotted as a function of time following head impact exposure, and utilized to provide both remove-from activity and safe return-to-activity guidance.

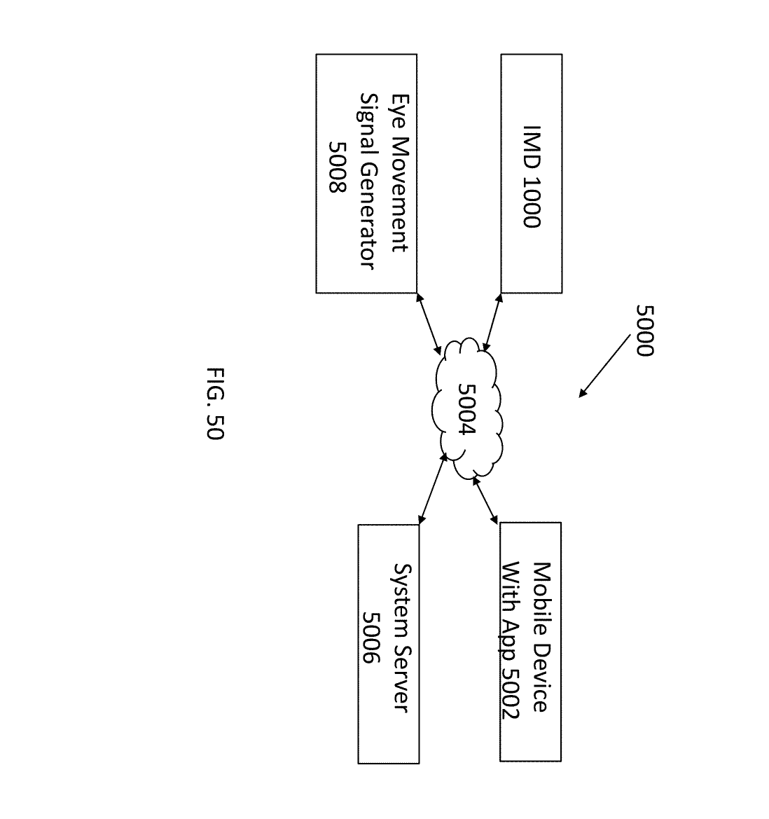

FIG. 50 illustrates a system 5000 configured in accordance with an embodiment of the invention. The system 5000 includes the disclosed IMD 1000 in communication with a mobile device operating an application 5002. Communication is via network 5004, which may be any combination of wired and wireless networks. The mobile device with application 5002 may perform some or many of the computations discussed in connection with IMD 1000 and communicate the results of such computations to the IMD 1000. Such an approach has an advantage of allowing for an IMD 1000 with less computational power and lower power consumption. Similarly, the mobile device with mobile application 5002 may communicate with a system server 5006 via network 5004. The system server 5006 may be used to perform computations otherwise performed by the IMD 1000 or the mobile device 5002. The results of such computations may be communicated directly to the IMD 1000 or may alternately by relayed to the IMD 1000 via mobile device with application 5002.

FIG. 50 also illustrates an eye movement signal generator 5008, which may be any of the devices shown in FIGS. 32, 35 and 36. Such devices communicate eye movement data via network 5004 to one or more of IMD 1000, mobile device with application 5002 and system server 5006. The corrected eye movement signals based upon sway data may be computed at one or more of IMD 1000, mobile device with application 5002 and system server 5006. Similarly, eye movement data indicative of physiological damage may be analyzed at one or more of IMD 1000, mobile device with application 5002 and system server 5006. The system server 5006 and/or mobile device with application 5002 may maintain databases of cumulative impact power data for many humans comprising individual teams and collections of teams. Similarly, such components may be used to store cumulative sway data, eye movement data, and gait measurement data.

While the preferred embodiment of the invention has been illustrated and described above, many changes can be made without departing from the spirit and scope of the invention, including use to monitor impact risks to many parts of the body and in many hazardous situations, use of many different methods of attachment to the body, and the integration of additional sensors to further aid in determining motion of the user, calculating values of other relevant injury biomarkers, or adjusting impact injury thresholds for remove-from-activity and return-to-activity guidance. Accordingly, the scope of the invention is not limited by the disclosure of the preferred embodiment. Instead, the invention should be determined entirely by reference to the claims that follow.

What is claimed is:

1. An apparatus, comprising:
a housing adapted for mechanical coupling with a skull of a human;
a first sensor positioned in the housing to collect linear motion signals;
a second sensor positioned in the housing to collect rotational motion signals; and
a processor positioned in the housing connected to the first sensor and the second sensor, the processor configured to
process the linear motion signals and the rotational motion signals to derive a cumulative impact power measure of repetitive sub-concussive head impacts,
compare the cumulative impact power measure to a threshold indicative of the onset of neural tissue deformations corresponding to physiological changes from repetitive sub-concussive head impacts, and
supply an alert when the cumulative impact power measure is proximate the threshold.

2. The apparatus of claim 1 wherein the housing has a first chamber for the first sensor, the second sensor, and the processor, a second chamber for a battery, and a flexible appendage connecting the first chamber and the second chamber.

3. The apparatus of claim 1 in combination with a collection of adhesive strips to mechanically couple the housing to the skull of the human.

4. The apparatus of claim 1 with an array of micro-structured polydimethylsiloxane pillars attached to the housing to operate as an adhesive to mechanically couple the housing and the skull of the human.

5. The apparatus of claim 4 wherein the array of micro-structured polydimethylsiloxane pillars terminate in suction-cups.

6. The apparatus of claim 1 wherein the processor is configured to execute a sway session during which the human wearing the housing is instructed to remain stationary for a specified period of time while linear motion signals and rotational motion signals are collected to form a current cumulative sway power measure.

7. The apparatus of claim 6 wherein the processor is configured to identify when the current cumulative sway power measure exceeds a threshold difference from base line sway data for the human indicative of physiological changes from repetitive sub-concussive head impacts.

8. The apparatus of claim 6 wherein the processor corrects eye movement signals using the current cumulative sway power measure to form corrected eye movement signals.

9. The apparatus of claim 8 wherein the processor is configured to identify when the corrected eye movement signals exceed a threshold difference from base line eye movement signals for the human indicative of physiological changes from repetitive sub-concussive head impacts.

10. The apparatus of claim 1 wherein the processor is configured to execute a gait measurement session during which the human wearing the housing is instructed to walk for a specified period of time while linear motion signals and rotational motion signals are collected to form current gait data.

11. The apparatus of claim 10 wherein the processor is configured to identify when the current gait data exceeds a threshold difference from base line gait data for the human indicative of physiological changes from repetitive sub-concussive head impacts.

12. The apparatus of claim 1 further comprising a wireless interface within the housing to communicate the cumulative impact power measure to a computation device with access to historical cumulative impact power measure data for the human.

13. The apparatus of claim 12 in combination with the computation device, wherein the computation device includes a memory storing instructions executed by a computation device processor to maintain a database of cumulative impact power measure data for a plurality of humans.

14. The apparatus of claim 12 in combination with the computation device, wherein the computation device includes a memory storing instructions executed by a computation device processor to maintain a database of sway signal data for a plurality of humans.

15. The apparatus of claim 12 in combination with the computation device, wherein the computation device includes a memory storing instructions executed by a computation device processor to maintain a database of gait data for a plurality of humans.

16. The apparatus of claim 12 in combination with the computation device, wherein the computation devices includes a memory storing instructions executed by a computation device processor to maintain a data base of eye movement data for a plurality of humans.

17. The apparatus of claim 12 in combination with the computation device, wherein the computation device includes a memory storing instructions executed by a computation device processor to produce download data and a computation device wireless interface to communicate the download data to the wireless interface of the apparatus.

18. The apparatus of claim 17 wherein the download data is selected from cumulative impact power measure data for the human, base line sway data for the human, base line eye movement for the human and base line gate data for the human.

19. The apparatus of claim 12 in combination with the computation device and an eye movement signal generation device.

20. The apparatus of claim 12 in combination with the computation device and a backend server in communication with the computation device.
